(19) United States
(12) Patent Application Publication
(10) Pub. No.: US 2025/0314614 A1
(43) Pub. Date: Oct. 9, 2025

(54) SEMICONDUCTOR SENSOR DEVICES, PACKAGING, FABRICATION AND USE-CASES

(71) Applicant: DiagMetrics, Inc., Madison, CT (US)

(72) Inventor: John James Daniels, Madison, CT (US)

(73) Assignee: DiagMetrics, Inc., Madison, CT (US)

(21) Appl. No.: 18/667,489

(22) Filed: May 17, 2024

Related U.S. Application Data
(63) Continuation-in-part of application No. 18/628,747, filed on Apr. 7, 2024.

(51) Int. Cl. G01N 27/414; G01N 27/12; G01N 33/543

(52) U.S. Cl. CPC G01N 27/4145; G01N 27/128; G01N 27/4148; G01N 33/5438

(57) ABSTRACT

The present invention relates to a novel packaging structure for a bare die semiconductor sensor, specifically designed for diagnostic applications where direct fluid sample contact with the sensor's detection area is required. The packaged semiconductor sensor comprises a semiconductor die with a top surface featuring bond pads and a detection area, and a bottom surface. A support member with a top side, a bottom side, and a detection window forms a sample well for receiving a fluid sample when aligned with the detection area. Z-axis conductive adhesive electrically connects bond pads to conductive traces on the support member. A sealing member seals the sample well, preserving sensor functionality while allowing the detection area exposure to the sample. This innovative packaging solution protects the sensor from environmental factors and maintains electrical integrity, enabling accurate and efficient biomarker detection in diagnostic procedures.

This application is a continuation-in-part of U.S. patent application Ser. No. 18/628,747, filed on 7 Apr. 2024, Inventor: John J. Daniels, the disclosure of which is incorporated herein in its entirety.

### BACKGROUND

This section is intended to provide a background or context to the exemplary embodiments of the invention as recited in the claims. The description herein may include concepts that could be pursued, but are not necessarily ones that have been previously conceived, implemented or described. Therefore, unless otherwise indicated herein, what is described in this section is not prior art to the description and claims in this application and is not admitted to be prior art by inclusion in this section.

The present invention relates generally to semiconductor sensors and, more particularly, to a unique packaging structure and manufacturing process for a bare die semiconductor sensor used in diagnostic and environmental applications, where a portion of the bare die sensor must remain exposed to allow a sample to be received at a detection area of the sensor.

In the field of semiconductor devices, including resistors, transistors, diodes, capacitors, and integrated circuits, the standard packaging approach is to fully encapsulate the semiconductor die to safeguard the internal semiconductor materials and device features. Traditional packaging solutions provide electrical connectivity through wire bonding that connects device features on the die with external pins or leads, which can then soldered onto a printed circuit board (PCB) or connected through a socket that is soldered on the PCB. This full encapsulation is considered essential for protecting the sensitive components from environmental factors that could compromise their integrity and functionality.

However, such packaging techniques are not optimal for semiconductor sensors designed to interact with a fluid sample for the detection of various analytes. These sensors require direct exposure of certain areas or the semiconductor device to the sample while still maintaining the integrity of the electrical connections and the sensor itself. There exists a need for a packaging structure that allows the sensor to function effectively in its intended diagnostic application.

Conventional packaging technologies face significant challenges when applied to diagnostic semiconductor sensors. Full encapsulation restricts access to the active area of the sensor device, preventing the necessary interaction with the fluid sample. This limitation has prompted the need for a new packaging approach that both protects the device and allows the fluid sample to contact the detection area of the sensor.

Moreover, the increasing demand for rapid, accurate, and point-of-care diagnostic tools necessitates the development of semiconductor sensors that can be incorporated into compact and user-friendly devices, which is impeded by traditional packaging methodologies.

This section is intended to provide a background or context to the exemplary embodiments of the invention as recited in the claims. The description herein may include concepts that could be pursued, but are not necessarily ones that have been previously conceived, implemented or described. Therefore, unless otherwise indicated herein, what is described in this section is not prior art to the description and claims in this application and is not admitted to be prior art by inclusion in this section.

The present invention relates generally to semiconductor sensors and, more particularly, to a unique packaging structure and manufacturing process for a bare die semiconductor sensor used in diagnostic and environmental applications, where a portion of the bare die sensor must remain exposed to allow a sample to be received at a detection area of the sensor.

In the field of semiconductor devices, including resistors, transistors, diodes, capacitors, and integrated circuits, the standard packaging approach is to fully encapsulate the semiconductor die to safeguard the internal semiconductor materials and device features. Traditional packaging solutions provide electrical connectivity through wire bonding that connects device features on the die with external pins or leads, which can then soldered onto a printed circuit board (PCB) or connected through a socket that is soldered on the PCB. This full encapsulation is considered essential for protecting the sensitive components from environmental factors that could compromise their integrity and functionality.

However, such packaging techniques are not optimal for semiconductor sensors designed to interact with a fluid sample for the detection of various analytes. These sensors require direct exposure of certain areas or the semiconductor device to the sample while still maintaining the integrity of the electrical connections and the sensor itself. There exists a need for a packaging structure that allows the sensor to function effectively in its intended diagnostic application.

Conventional packaging technologies face significant challenges when applied to diagnostic semiconductor sensors. Full encapsulation restricts access to the active area of the sensor device, preventing the necessary interaction with the fluid sample. This limitation has prompted the need for a new packaging approach that both protects the device and allows the fluid sample to contact the detection area of the sensor.

Moreover, the increasing demand for rapid, accurate, and point-of-care diagnostic tools necessitates the development of semiconductor sensors that can be incorporated into compact and user-friendly devices, which is impeded by traditional packaging methodologies.

### BRIEF SUMMARY

The invention described herein addresses these challenges by providing a packaged semiconductor sensor with a novel structure that includes an open detection area for fluid sample contact. This configuration enables the semiconductor sensor to analyze samples effectively while ensuring that the rest of the semiconductor die is adequately protected and the electrical connections are maintained.

In accordance with an aspect of the invention, a packaged semiconductor sensor includes a semiconductor die having a top surface and a bottom surface, with at least two bond pads and at least one detection area located at the top surface. A support member has a top side and a bottom side, and a detection window provided as an opening in the support member from the top side to the bottom side. The opening/detection window in the support member and the detection area located at the top surface of the semiconductor die define a sample well for receiving a sample to be tested by the packaged semiconductor sensor. A least two conductive traces are provided on the bottom side of the support member. A z-axis conductive adhesive bonds and electrically connects a respective one of the bond pads to a corresponding one of the conductive traces. A sealing member seals the bottom side of the support member with the top surface of the die to seal the sample well. The z-axis conductive adhesive can also be used to form the sealing member. Alternatively or additionally, the sealing member can comprise at least one of an epoxy, glue, pressure sensitive adhesive and gasket.

In accordance with another aspect of the invention, a biosensor card assembly includes a bare die semiconductor sensor with a top surface including two or more bond pads and at least one detection area. A support member has at least a corresponding number of conductive traces as the bond pads on the bare die. The conductive traces are provided on at least a bottom side of the support member for connecting with the bond pads of the bare die. The support member has a through-hole detection window aligning with the detection area of the bare die. A conductive adhesive is provided between each bond pad of the bare die and a corresponding conductive trace of the support member. The conductive epoxy provides an electrical connection between a respective bond pad and a corresponding conductive trace.

In accordance with another aspect of the invention, a sensor card assembly is configured for enhanced fluid sample analysis. A sensor element is provided with a detection area and a plurality of bond pads on a top surface. The support member includes a top side with integrated liquid detection features and a bottom side providing conductive traces corresponding to the bond pads. A z-axis conductive adhesive is provided between each bond pad and a corresponding conductive trace for selective electrical connection in the z-axis direction. A detection window on the top side of the support member is aligned with the detection area of the sensor element to form a sample well.

In accordance with another aspect of the invention, an integrated biosensor card and bare die sensor assembly is provided for targeted biomarker detection. A semiconductor die has a top surface with a least one sensor device and at least one sensor area and bond pads associated with each sensor device. A support member having a bottom side with conductive traces corresponding to the bond pads supports the semiconductor die and connects the semiconductor devices of the die to a printed circuit board. An accumulator in fluid communication with the sensor areas applies an electrostatic field to a fluid sample for aligning target biomarkers within a fluid sample. The accumulator facilitates enhanced detection by modulating the orientation and proximity of target biomarkers to the immobilized capture molecules at the sensor areas.

The invention described herein addresses these challenges by providing a packaged semiconductor sensor with a novel structure that includes an open detection area for fluid sample contact. This configuration enables the semiconductor sensor to analyze samples effectively while ensuring that the rest of the semiconductor die is adequately protected and the electrical connections are maintained.

In accordance with an aspect of the invention, a packaged semiconductor sensor includes a semiconductor die having a top surface and a bottom surface, with at least two bond pads and at least one detection area located at the top surface. A support member has a top side and a bottom side, and a detection window provided as an opening in the support member from the top side to the bottom side. The opening/detection window in the support member and the detection area located at the top surface of the semiconductor die define a sample well for receiving a sample to be tested by the packaged semiconductor sensor. A least two conductive traces are provided on the bottom side of the support member. A z-axis conductive adhesive bonds and electrically connects a respective one of the bond pads to a corresponding one of the conductive traces. A sealing member seals the bottom side of the support member with the top surface of the die to seal the sample well. The z-axis conductive adhesive can also be used to form the sealing member. Alternatively or additionally, the sealing member can comprise at least one of an epoxy, glue, pressure sensitive adhesive and gasket.

In accordance with another aspect of the invention, a biosensor card assembly includes a bare die semiconductor sensor with a top surface including two or more bond pads and at least one detection area. A support member has at least a corresponding number of conductive traces as the bond pads on the bare die. The conductive traces are provided on at least a bottom side of the support member for connecting with the bond pads of the bare die. The support member has a through-hole detection window aligning with the detection area of the bare die. A conductive adhesive is provided between each bond pad of the bare die and a corresponding conductive trace of the support member. The conductive epoxy provides an electrical connection between a respective bond pad and a corresponding conductive trace.

In accordance with another aspect of the invention, a sensor card assembly is configured for enhanced fluid sample analysis. A sensor element is provided with a detection area and a plurality of bond pads on a top surface. The support member includes a top side with integrated liquid detection features and a bottom side providing conductive traces corresponding to the bond pads. A z-axis conductive adhesive is provided between each bond pad and a corresponding conductive trace for selective electrical connection in the z-axis direction. A detection window on the top side of the support member is aligned with the detection area of the sensor element to form a sample well.

In accordance with another aspect of the invention, an integrated biosensor card and bare die sensor assembly is provided for targeted biomarker detection. A semiconductor die has a top surface with a least one sensor device and at least one sensor area and bond pads associated with each sensor device. A support member having a bottom side with conductive traces corresponding to the bond pads supports the semiconductor die and connects the semiconductor devices of the die to a printed circuit board. An accumulator in fluid communication with the sensor areas applies an electrostatic field to a fluid sample for aligning target biomarkers within a fluid sample. The accumulator facilitates enhanced detection by modulating the orientation and proximity of target biomarkers to the immobilized capture molecules at the sensor areas.

In one aspect, a method, includes the steps of i) Providing a semiconductor wafer having a transparent substrate, ii) Forming device regions each includes a source, a drain and at least one channel region, iii) Forming a gate oxide layer over each channel region, iv) Forming a detection area area including a charge transfer layer over the gate oxide layer, v) Immobilizing capture molecules on the charge transfer layers, includes the steps of a) immobilizing at least a first set of activatable linker molecules and a second set of activatable linker molecules on the charge transfer layer of device region, each respective first set and second set of activatable linker molecules being activated for binding by a different corresponding first wavelength of linker-activating radiation and second wavelength of linker-activating radiation, and b) disposing over a surface of the semiconductor substrate wafer covering the plurality of device regions a capture molecule carrier fluid containing at least a first set of activatable capture molecules and a second set of activatable capture molecules as free-floating activatable capture molecules, each respective first set and second set of activatable capture molecules being activated for binding by a different corresponding first wavelength of capture molecule-activating radiation and second wavelength of capture molecule-activating radiation. The method also includes c) selectively irradiating through the transparent substrate a first pattern of radiation includes the first wavelength of linker-activating radiation and the first wavelength of capture molecule-activating radiation to bind a first set of activated capture molecules to a first set of activated linker molecules. The method also includes d) selectively irradiating through the transparent substrate a second pattern of radiation includes the second wavelength of linker-activating radiation and the second wavelength of capture molecule-activating radiation to bind a second set of activated capture molecules to a second set of activated linker molecules.

In one aspect, a semiconductor sensor system includes a semiconductor sensor having a source, a drain, and a substrate configured to form a two-dimensional electron gas (2DEG) within a detection area of the sensor, capture molecules immobilized within the detection area for detecting target molecules, an Exhaled Breath Condensate (EBC) Collector configured as a chilled thermal mass connected to the semiconductor sensor, a thermally conductive adhesive directly coupling the semiconductor sensor to the EBC Collector to facilitate heat transfer from the semiconductor sensor to the EBC Collector, and where the EBC Collector is configured to stabilize the temperature of the semiconductor sensor during operation, thereby reducing thermal effects that cause non-linear sensor responses and charge trapping.

In one aspect, a semiconductor sensor system includes a substrate, a detection area located on the substrate configured to include capture molecules for detecting target molecules, a source and a drain disposed on the substrate and forming part of a two-dimensional electron gas (2DEG) system for measuring electrical properties affected by interactions within the detection area, an insulator coupled to the substrate via an adhesive, configured to thermally isolate the semiconductor sensor from external thermal effects during operation, where the semiconductor sensor further includes a Device Under Test (DUT) sensor and a reference sensor, the DUT sensor being exposed to a liquid sample containing target molecules and the reference sensor being unexposed to the liquid sample to minimize thermal effects influenced by the electrical current flow from the source to the drain.

In one aspect, a sensor system includes a substrate equipped with a detection area, capture molecules immobilized within the detection area for binding target molecules, a source and a drain arranged on the substrate to form a flow of charges between the source and the drain dependent on the binding between the capture molecules and the target molecules, and top and bottom driving electrodes configured to apply an electric field to orient and migrate molecules within a sample towards or away from the detection area.

In one aspect, a method for fabricating a multi-biomarker detecting semiconductor sensor array, includes i) Providing a semiconductor wafer having a transparent substrate. The method also includes ii) Forming and array of sensor devices each includes a source, a drain, and at least one channel region. The method also includes iii) Forming a gate oxide layer over each channel region. The method also includes iv) Forming an array of detection areas including the gate oxide layer. The method also includes v) Immobilizing capture molecules on the detection areas through a multi-step process, includes a) Immobilizing a first set of activatable linker molecules on the detection area of each device region, each linker molecule being activatable by a specific wavelength of linker-activating radiation, b) Applying a first capture molecule carrier fluid over the surface of the semiconductor substrate wafer covering the plurality of device regions, the fluid containing a first set of capture molecules as free-floating activatable capture molecules, c) Selectively irradiating through the transparent substrate with a specific pattern of radiation corresponding to the activating wavelength for the first set of linker molecules, binding the first set of capture molecules to the activated linker molecules, d) Removing the first capture molecule carrier fluid and rinsing the surface to leave behind the immobilized first set of capture molecules, e) Optionally repeating steps b) through d) for subsequent sets of capture molecules, each set being provided in a new carrier fluid and activated for binding by a different corresponding wavelength of radiation, and f) selectively irradiating with subsequent patterns of radiation for each subsequent set of capture molecules, facilitating the selective immobilization on the charge transfer layers.

In one aspect, a method of fabricating a multi-biomarker detecting array of semiconductor sensors on a bare die, includes Providing a semiconductor substrate, Forming a plurality of drain electrodes on the substrate, Depositing a drain insulation layer over the drain electrodes, Forming a plurality of source electrodes orthogonal to the drain electrodes and over the drain insulation layer, Depositing a barrier layer over the source and drain electrodes, leaving an opening for forming an array of detection areas, Defining a glass layer over the barrier layer to form sample wells at each detection area while exposing bond pads for the source and drain electrodes, and Forming a gate insulation layer over the glass layer, configuring the detection areas to function as field effect transistors with individually addressable source and drain electrodes.

In one aspect, a vertical GaN semiconductor sensor includes a N+GaN wafer forming a substrate, a N−GaN drift layer disposed over said N+GaN wafer, a plurality of capillary channels defined within said N−GaN drift layer, a pGaN layer disposed over said N−GaN drift layer, forming p-n junctions therewith, a drain located at a bottom portion of said N+GaN wafer, multiple sources disposed at a top surface of said pGaN layer, a plurality of depletion layers formed at interfaces between said pGaN layer and said N−GaN drift layer, configured to control charge carrier flow based on binding events occurring at detection areas, capture molecules immobilized within said capillary channels and configured to bind target molecules, a liquid gate electrode configured to apply a gate voltage through a liquid sample disposed at said detection areas, where said sensor is configured to detect target molecules by modulation of the depletion layers and charge carrier flow in response to binding events.

In one aspect, a method for detecting molecular binding events using a Wheatstone Bridge circuit, includes arranging a plurality of semiconductor sensors, each having a source and a drain, to form a Wheatstone Bridge circuit where each sensor's source-to-drain resistance serves as one of the resistors in the bridge, connecting the Wheatstone Bridge to a power source and a voltage measurement device across two output terminals, balancing the Wheatstone Bridge such that the voltage across the output terminals is zero under a baseline condition without target molecule binding, exposing the semiconductor sensors to a sample potentially containing target molecules, and detecting a voltage difference across the output terminals, the voltage difference being indicative of a molecular binding event that alters the source-to-drain resistance of at least one sensor in the bridge.

### BRIEF DESCRIPTION OF THE SEVERAL VIEWS OF THE DRAWINGS

FIG. 1 is a partial exploded perspective view of the biosensor card components, illustrating the spatial relationship between the conductive traces, the z-axis conductive adhesive, and the bare die semiconductor sensor.

FIG. 2 illustrates a top-down view of the biosensor card, showing the device features on the top surface of the bare die semiconductor sensor.

FIG. 3 is an exploded view diagram showing the layered construction of the biosensor card showing the separate components starting from the top overlay, with a liquid detection window, followed by the top conductive traces, the detection window on the support member, and the bottom conductive traces through to the bottom overlay.

FIG. 4 is a top-down, layer-by-layer view of the biosensor card's material stack.

FIG. 5 illustrates the material composition and stack-up dimensions for an etched copper flex circuit version of the biosensor card.

FIG. 6 shows a top view of a bare die semiconductor sensor, showing the layout of individual source and gate connections for four sensor devices, with a common drain.

FIG. 7 is a cross-sectional view of a GaN biosensor, showing the layered structure with a detection well at the top surface.

FIG. 8 shows top and bottom views of the fully assembled biosensor card.

FIG. 9 illustrates a semiconductor biosensor with six individually addressable GaN HEMT (High Electron Mobility) sensor devices sharing a common gate and drain.

FIG. 10 shows an alternative semiconductor sensor design with five functional test devices and one reference device.

FIG. 11 is a logic flow diagram for Applied Probabilistic Analysis to determine the detecting of a target biomarker, and illustrates the operation of an exemplary method utilized by an exemplary diagnostic device and electronic medical records database.

FIG. 12 shows the die attachment process for a bare die semiconductor sensor to the support member.

FIG. 13 shows an isolated and partial assembly view of an embodiment of the biosensor card showing the arrangement of conductive traces, detection areas, sample wells, and other relevant structural features of a biosensor card assembly with four individually addressable and functionalizable sensor devices on a single bare die.

FIG. 14 illustrates an embodiment of a completed biosensor card with four individually addressable sensor devices.

FIG. 15 shows the top side and bottom side of a biosensor card with four independently functional sensor devices, each with its own sample well for targeted molecule detection within the same fluid sample.

FIG. 16 is an isolated view showing a bare die sensor attached to conductive traces.

FIG. 17 shows a biosensor card with attached sensor die, microfluidic liquid sample flow path and compressed cellulose wick, where the biosensor card assembly is electrically and mechanically connected with a printed circuit board.

FIG. 18 is a close up view of a bare die sensor attached to a biosensor card with the detection window of the biosensor card aligned with the detection area of the sensor.

FIG. 19 shows a semiconductor sensor device with an accumulator to selectively position target molecule in a fluid sample for enhanced biomarker detection.

FIG. 20 is a side view of a sensor for detecting target molecule in a fluid sample with a detection area for receiving the fluid sample and a top driving electrode and a bottom driving electrode where an electric potential drives the target molecule towards the capture molecules to concentrate the target molecule in a portion of the fluid sample received at the detection area.

FIG. 21 is a flowchart of the steps for concentrating the target molecule in a fluid sample and testing for a change in electrical characteristics of a functionalized transistor sensor.

FIG. 22 illustrates a graphene detection interface with nanoCLAMP capture molecules immobilized by linker molecules, with a portion of the capture molecules immobilized at a greater distance from the detection interface than another portion of the capture molecules.

FIG. 23 illustrates target and non-target molecules free floating in a liquid medium in the vicinity of capture molecules immobilized on a graphene detection surface.

FIG. 24 illustrates an electric field potential applied in the detection area and driving a target molecule and non-target molecule towards the capture molecules, where the target molecule is captured by a capture molecule extending on linker molecules at a relatively longer distance from the detection interface.

FIG. 25 illustrates the electric field potential removed from detection area.

FIG. 26 illustrates an opposite polarity electric field potential that drives the target molecule and non-target molecule away from the detection interface, where the target molecule remains tethered by the capture molecule immobilized by the relatively long linker on the detection interface.

FIG. 27 illustrates more target molecule and non-target molecules flowing into the detection area.

FIG. 28 illustrates the electric field potential applied in the detection area, driving the tethered target molecule into position to be captured by a capture molecule immobilized by a relatively shorter linker molecule, and where another target molecule is captured by another capture molecule extending on linker molecules at a relatively longer distance from the detection interface to concentrate the target molecules in a portion of the fluid sample received at the detection interface.

FIG. 29 shows a semiconductor sensor device fixed to a large thermal mass, such as the EBC Collector, through a conductive adhesive to remove internally generated heat from the sensor during operation.

FIG. 30 shows a semiconductor sensor device fixed to an insulator to thermally isolate the sensor during operation.

FIG. 31 stylistically illustrates a field of capture molecules immobilized on the detection area of a sensor device with a single molecule that is about to bind with one or more of the capture molecules.

FIG. 32 stylistically illustrates a target molecule binding to one or more capture molecules showing a propagation of charge effects radiating through the field of capture molecules.

FIG. 33 shows the formation of an array of drain conductors of a reconfigurable bare die semiconductor sensor.

FIG. 34 shows the formation of a drain insulation layer on the an array of drain conductors of the reconfigurable bare die semiconductor sensor.

FIG. 35 shows the formation of an array of source conductors at a right angle to the array of drain conductors and over the drain insulation layer of the reconfigurable bare die semiconductor sensor.

FIG. 36 shows a barrier layer formed over the arrays of drain and source conductors and having an opening for forming an array of detection areas.

FIG. 37 shows a detection area field formed over the arrays for drain and source conductors of the reconfigurable bare die semiconductor device.

FIG. 38 shows a pair of drain conductors and a pair of source conductors being tapped for electrical measurement of the source to drain current flow.

FIG. 39 illustrates the layout of a sensor array with a glass passivation layer, showcasing individually addressable detection areas, test points for electrical signal capture, and a schematic for AI-driven analysis of binding event patterns.

FIG. 40 illustrates a sensor array having test points for signal extraction, a measured binding event, and a liquid gate electrode.

FIG. 41 illustrates a sensor array with 120 individually addressable sensor devices featuring a structured layout of twelve drain electrodes.

FIG. 42 depicts a biosensor card featuring an integrated antenna for wireless communication, liquid detection capabilities, and a bare die biosensor array with 120 individually addressable devices customizable for either panel tests with different capture molecules or a wide area FET sensor with uniform functionalization to enhance detection sensitivity.

FIG. 43 illustrates bare die die biosensor having semiconductor sensor devices functionalized for FluA/FluB/SARS testing.

FIG. 44 illustrates an applied-field-reactive capture molecule conjugate having an applied-field-responsive end and a capture molecule end with a linker molecule providing electro-chemical properties that change at least one of a polarity and a conductivity.

FIG. 45 illustrates a capture molecule conjugate having a magnetically attractive end and a capture molecule conjugate end.

FIG. 46 shows a process for forming aligned and oriented capture molecule conjugates aligned in an aligning field on a dissolvable adhesive.

FIG. 47 shows a process for forming aligned and oriented capture molecule conjugates aligned in an electric field on a dissolvable adhesive.

FIG. 48 illustrates a bare die sensor having a detection area covered by a field of capture molecules, and a liquid sample containing specific target molecules and non-specific ions and molecules.

FIG. 49 illustrates electric field applying top and bottom driving electrodes applying an electric field causing the specific target molecules and non-specific ions and molecules in the liquid sample to orient and migrate in a direction depending on the positive and negative charges of the molecules and ions, where the capture molecules and target molecules are brought into the potential for a specific binding event.

FIG. 50 shows the applied electric field being reversed causing the molecules and ions to orient and migrate in another direction where the target molecules that bind with the capture molecules remain immobilized at the detection area.

FIG. 51 shows an optional step of washing away non-specific ions and molecules that are not bound to capture molecules to improve the signal caused by target molecules that remain immobilized at the detection area.

FIG. 52 illustrates different capture molecule for detecting different target molecules and bonding to activatable linkers.

FIG. 53 shows three sensors formed on a wafer having a transparent substrate, with a combination of selectively activate-able capture molecule and linker pairs.

FIG. 54 illustrates the steps of selectively binding a first sub-set of capture molecules to activatable linker molecules.

FIG. 55 illustrates illustrates the steps of selectively binding a respective second and third sub-set of capture molecules to corresponding activatable linker molecules.

FIG. 56 shows three sensors formed on a wafer having a transparent substrate, each respective sensor having a corresponding selectively activate-able capture molecule and linker pair immobilized on its detection area.

FIG. 57 illustrates an emission plate with individually addressable emitting pixels for selectively immobilizing capture molecules on respective detection areas of a biosensor array.

FIG. 58 shows a Wheatstone Bridge circuit concept utilizing semiconductor sensors, where each sensor's source-to-drain resistance acts as a bridge resistor, enabling the detection of molecular binding events that cause measurable changes in resistance in the S-D resistance of a DUT (device under test).

FIG. 59 displays a vertical GaN sensor array in its baseline state, showing the detailed arrangement of components like depletion layers, drains, gates, and sources, set up for detecting molecular interactions.

FIG. 60 illustrates a vertical GaN sensor system where target molecule binding to capture molecules within the device's structure causes changes in the depletion layer, facilitating enhanced charge conduction and molecule detection.

FIG. 61 depicts a vertical GaN semiconductor sensor enhanced with liquid gate electrodes, which apply a gate voltage through a liquid sample to dynamically adjust the depletion layer and improve the detection of chemical or biological entities.

FIG. 62 illustrates a vertical GaN sensor with a liquid gate, showing how target molecules in a liquid sample influence the sensor's detection areas by binding with capture molecules, thus modulating electrical properties for enhanced signal detection.

### DETAILED DESCRIPTION

Below are provided further descriptions of various non-limiting, exemplary embodiments. The below-described exemplary embodiments are separately numbered for clarity and identification. This numbering should not be construed as wholly separating the below descriptions since various aspects of one or more exemplary embodiments may be practiced in conjunction with one or more other aspects or exemplary embodiments. That is, the exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The foregoing and other aspects of exemplary embodiments of this invention are made more evident in the following Detailed Description, when read in conjunction with the attached Drawing Figures, wherein:

Below are provided further descriptions of various non-limiting, exemplary embodiments. The below-described exemplary embodiments are separately numbered for clarity and identification. This numbering should not be construed as wholly separating the below descriptions since various aspects of one or more exemplary embodiments may be practiced in conjunction with one or more other aspects or exemplary embodiments. That is, the exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The foregoing and other aspects of exemplary embodiments of this invention are made more evident in the following Detailed Description, when read in conjunction with the attached Drawing Figures, wherein:

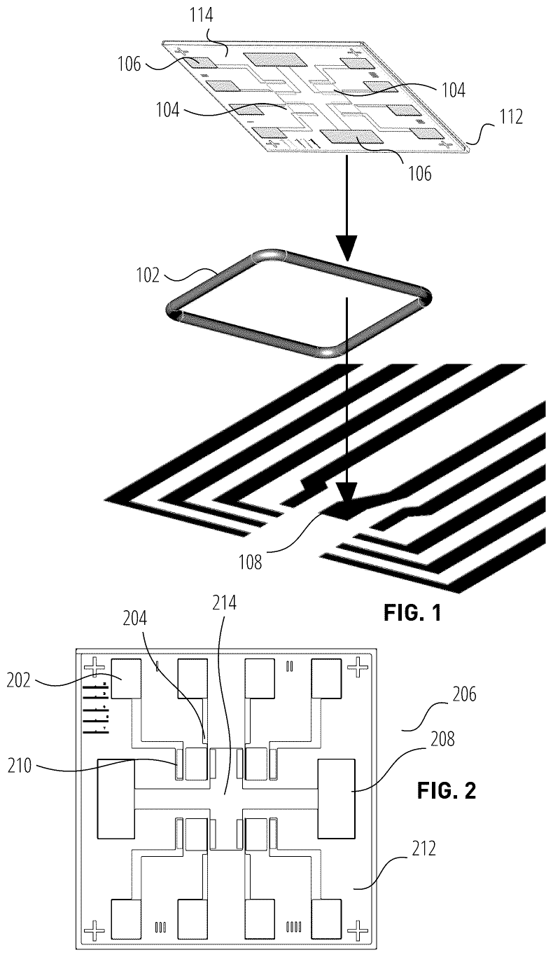

FIG. 1 shows a partial exploded view of a semiconductor biosensor card, showing the assembly and arrangement of the main conductive components. An exemplary embodiment comprises a sealing member 102, sensor devices 104, bond pads 106, conductive traces 108, a bottom surface 112, and a top surface 114.

Positioned to be attached to the bottom conductive traces is the semiconductor die. This bare die includes GaN HEMT, g-FET, or other semiconductor elements that provide a biological or environmental sensor for the detection of targeted analytes. The top surface of the semiconductor die includes the bond pads and the detection area. The bond pads are connected to the conductive traces via a z-axis conductive adhesive, which provides both mechanical attachment and electrical continuity. In an exemplary embodiment, the z-axis conductive adhesive also provides a sealing member the seals a sample well defined by the top surface of the die and the walls of the detection window. Alternatively, additional layers can be formed to increase the volume of the sample well. Also, alternatively, the sealing member can be formed using an additional bead of a non-conductive material such as silicone.

The z-axis conductive adhesive allows for vertical (z-axis) electrical connection from the bond pads on the semiconductor die to the conductive traces on the support member, without shorting between conductive traces and/or bond pads. The z-axis conductive adhesive has an anisotropic conductivity profile that prevents lateral electrical connectivity, providing signal transmission from the detection area while also providing a reliable electrical bond, mechanical attachment and a fluid seal, without interfering with the detection area on the top surface of the die. An example of a z-axis conductive adhesive is an anisotropic conductive adhesive 125-01A/B-187 from Creative Materials, Ayer, MA. The conductive adhesive is applied, for example, using a conventional die bonder semiconductor processing equipment so that after dispensing the adhesive onto the conductive traces and a pick and place operation, the adhesive is between each bond pad of the bare die and corresponding conductive trace of the support member.

The packaged semiconductor sensor includes a semiconductor die having a top surface and a bottom surface, with at least two bond pads and at least one detection area located at the top surface. A support member has a top side and a bottom side, and a detection window provided as an opening in the support member from the top side to the bottom side. The opening/detection window in the support member and the detection area located at the top surface of the semiconductor die define a sample well for receiving a sample to be tested by the packaged semiconductor sensor. A least two conductive traces are provided on the bottom side of the support member. Each bond pad of the bare die is aligned with and electrically connected to a respective conductive trace via the z-axis conductive epoxy without the need for wire bonding.

A z-axis conductive adhesive bonds and electrically connects a respective one of the bond pads to a corresponding one of the conductive traces. A sealing member seals the bottom side of the support member with the top surface of the die to seal the sample well. The z-axis conductive adhesive can also be used to form the sealing member. Alternatively or additionally, the sealing member can comprise at least one of an epoxy, glue, pressure sensitive adhesive and gasket.

The detection window/opening and the detection area collectively define a sample well for receiving a fluid sample to be analyzed. A sealing member can integrated or formed separately with the z-axis conductive epoxy that seals the sample well. For example, the sealing member can be composed of the z-axis conductive epoxy, and/or the sealing member can be composed of a non-conductive adhesive that seals the detection area and detection window to form the sample well and provide a barrier to protect the z-axis conductive adhesive from contacting a fluid disposed in the sample well.

FIG. 2 shows a top-down view of a semiconductor biosensor, detailing the surface features integral to the functionality as a sensor including features at the top surface that are involved in the detection of specific analytes. An exemplary embodiment comprises a bond pad 202, a gate 204, a die 206, a drain 208, a source 210, and a top side 212. At the center of the image is the detection area, a key functional part of the biosensor where the capture molecules are immobilized for selective binding and detection of target molecule present in a fluid sample. The capture molecules at the detection area interact with the fluid sample directly and therefore the detection area at the top surface of the packaged semiconductor sensor must remain open for receiving the sample.

Surrounding the detection area are bond pads, which are small, conductive areas on the top surface of the bare die. Each bond pad is positioned to interface with corresponding conductive traces on the support member to provide the transmission of changes in electrical signals resulting from the detection events (capture molecule/target molecule binding) that occurs at or near the detection area the top surface of the die.

The sensor devices on the bare die includes gate, source and drain features connected with the bond pads. The layout of the bare die features are selected to enable the electrical connections to the conductive traces via the z-axis conductive adhesive while ensuring the detection area remains unobstructed for sample interaction.

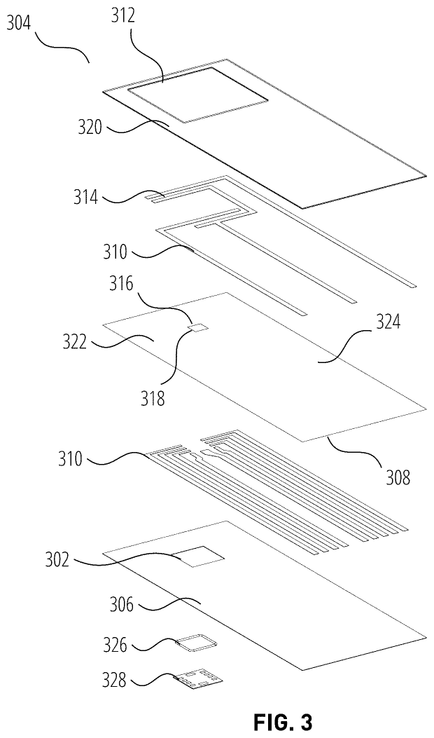

FIG. 3 shows an exploded view of the layered structure of the biosensor card, providing a detailed illustration of each layer and its respective components as they would be assembled in the manufacturing process. An exemplary embodiment comprises a bare die window 302, a biosensor card 304, a bottom overlay 306, a bottom side 308, a conductive trace 310, a detection window 312, liquid detection features 314, a liquid detection window 316, an opening 318, a top overlay 320, a support member 322, a top side 324, a z-axis conductive adhesive 326, and a bare die 328.

A top overlay is the uppermost layer of the biosensor card. Directly beneath the top overlay are the conductive traces that form liquid detection features, in this exemplary embodiment the conductive traces are etched copper on a flexible substrate material such as Kapton. At least one liquid detection feature is integrated into the top surface of the support member to monitor presence of a fluid sample.

A detection window is formed as an opening within the support member. The detection window aligns with the detection area of the semiconductor die, enabling the fluid sample to contact the biosensor's detection region.

The bottom side of the support member has another set of conductive traces. These traces on the bottom side provide electrical connections to the corresponding bond pads on the semiconductor die.

Starting from the top of the figure, the first layer is the top overlay, which functions as a protective cover for the underlying components. This overlay features a liquid detection window that allows the liquid sample to reach the liquid detection features. The liquid detection features are used for determining the flow of the a liquid sample before and after the sample is received by the detection area of the biosensor.

Below the top overlay, the top conductive traces are shown. The conductive traces can be etched copper or printed conductive ink and provide the electrical pathways on the flexible substrate support member to provide electrical connectivity to the sensor elements.

The support member layer includes a detection window, an aperture that is aligned with the detection area on the semiconductor die and defines along with the detection area the sample well where the fluid sample is collected and analyzed.

The support member provides a platform onto which the semiconductor die will be attached. The support member includes bottom conductive traces on its reverse side, which are in turn connected to external electronic circuitry and provide a means for electrical signals to be read out from the sensor.

A bottom overlay protects the bottom side of the biosensor card, completing the assembly. The bottom overlay includes a bare die window that provides an opening to the bottom conductive traces for connecting with the bond pad of the bare die via the z-axis conductive adhesive.

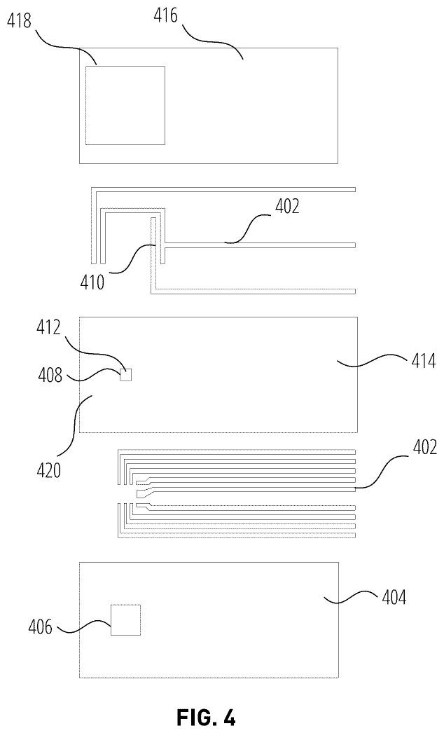

FIG. 4 shows a layered breakdown of the biosensor card, depicted in a top-down view. This drawing illustrates each component of the material stack to indicate the layout and individual function of the layers that form the complete biosensor card. An exemplary embodiment comprises a conductive trace 402, a bottom overlay 404, a bare die window 406, a detection window 408, a liquid detection features 410, an opening 412, a support member 414, a top overlay 416, a liquid detection window 418, and a top side 420.

The topmost layer shown is the top overlay, which acts as the protective outer covering of the biosensor card. It features a predefined liquid detection window that corresponds to the location of the underlying liquid detection features. The liquid detection features are formed on the top side and operative to indicate a fluid sample presence and flow characteristics. Below the top overlay is the layer of top conductive traces that form the liquid detection features.

The support member has the conductive traces formed thereon either by a subtractive process, such as etching copper foil, or by an additive process, such as screen printing conductive silver ink. The support member also include the detection window that aligns with the detection area on the semiconductor die, allowing the biosensor to interact with the test sample.

Beneath the support member, bottom conductive traces connect the die sensor devices features with an external electronic circuit. The bottom overlay protects and insulates the bottom conductive traces and includes the bare die window for connecting the bare die with the conductive traces.

The support member can be one of a flex circuit having an etched metal pattern forming the conductive traces, a plastic substrate having printed conductive ink forming the conductive traces, and rigid circuit board having at least one of etched metal and printed conductive ink forming the conductive traces.

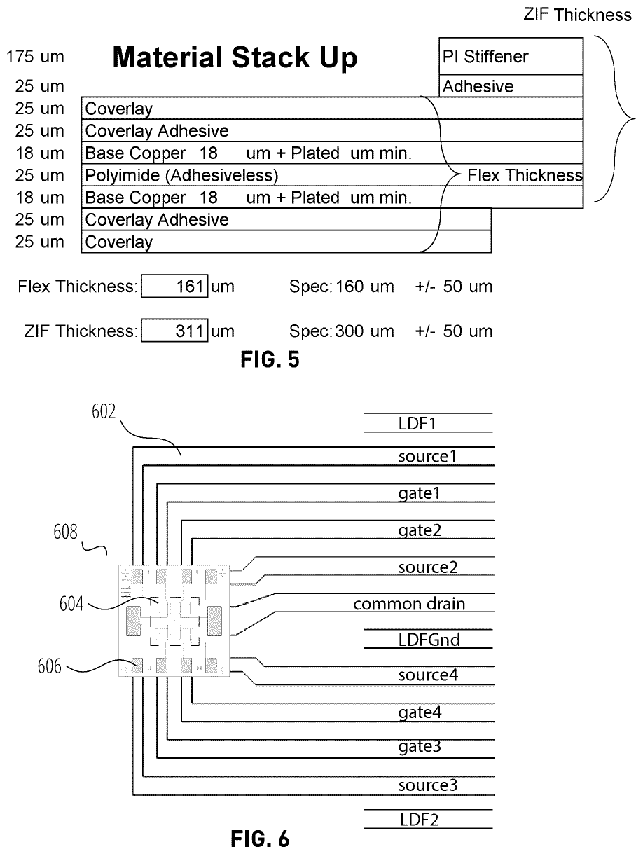

FIG. 5 shows the material stack-up for a biosensor card, specifically designed in this exemplary embodiment as an etched copper flex circuit. This drawing illustrates the various layers and their respective thicknesses for forming the biosensor card using conventional and well-known manufacturing processing and materials. The materials and thickness as for example only, other constructions can also be used. For example, a polyester (PET) or other suitable plastic substrate may be used with screen printed conductive ink forming the conductive traces. This plastic substrate embodiment may be particularly advantageous since the additive manufacturing process will be lower cost and have less environmental impact that the use of an etched copper flex circuit construction. The die attach process can be done at relatively lower temperatures, for example, using a UV curable z-axis conductive adhesive or tape, making the lower cost plastic substrate with screen printed conductive ink an attractive alternative to etch copper on Kapton.

The stack-up begins with the top overlay or coverlay, a protective layer measuring 25 micrometers (μm) in thickness that protects the underlying circuitry. A layer of overlay adhesive of the same thickness secures the top overlay to the base copper layer and the support member. The base copper layer, which is 18 μm thick, forms the conductive pathways for the circuit and includes additional thickness from plating, ensuring robust electrical connections. Following the copper layer, a 25 μm thick adhesiveless polyimide layer forms the substrate of the support member. Another etched copper layer of 18 μm plus plating forms the bottom conductive traces.

To complete the stack, an additional overlay adhesive and a bottom overlay layer, each 25 μm thick, help to encapsulate and protect the entire assembly. For dimensional stability and to interface with an electronic circuit connector, such as ZIF connector, a stiffener with adhesive is incorporated at the edge of the biosensor card, contributing to a total ZIF connector end thickness of 311 μm.

FIG. 6 shows a detailed top view of a multi-sensor GaN biosensor device with four individually addressable sensor elements, each with its own source and gate connections and sharing a common drain. The drawing illustrates the design and layout of the sensor's ohmic features that form the bond pads on the top surface of the bare die. An exemplary embodiment comprises a conductive trace 602, a detection area 604, a bond pad 606, and a die 608.

A detection area is formed at the central region of the die, but could be formed at other locations and the die could also include other electronic features connected with features of the sensor devices. For example, resistor, capacitors, transistors and other semiconductor electronic devices can be provided directly on the die and/or provided as discrete electronic devices provided on a printed circuit board connected with the sensor devices through the biosensor card. Capture molecules are immobilized at the detection area that are specific to the target analyte(s). The detection area is positioned to be in direct contact with the sample in the detection well.

Surrounding the detection area, the bare die features various bond pads, which serve as the terminals for electrical connectivity. The bond pads are connected via the z-axis conductive adhesive to the conductive traces and form the electrical pathways for signal detection.

The conductive traces are laid out to provide electrical connections to the designated source and gate terminals for each of the four sensor devices provided on the exemplary bare die. These traces are labeled for clarity, with “source1” through “source4” indicating the source connections for each sensor and “gate1” through “gate4” for the gate connections. The “common drain” trace provides a streamlined and simplified electronic circuit.

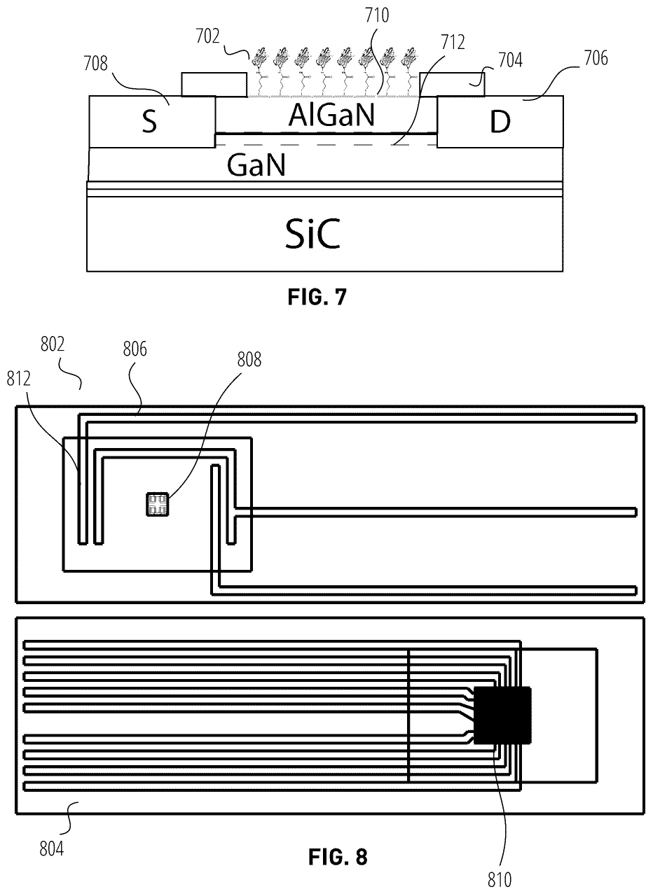

FIG. 7 provides a cross-sectional representation of a Gallium Nitride (GaN) biosensor, illustrating the essential components and their arrangement within the device. This detailed depiction is instrumental for understanding the biosensor's functional design, particularly its capability to detect target molecule. An exemplary embodiment comprises capture molecules 702, a sample well 704, a drain 706, a drain 708, a detection area 710, and a 2DEG 712.

In this exemplary embodiment, the substrate of the structure is a Silicon Carbide (SiC) wafer. Above the SiC substrate, the primary functional layer of the GaN is formed. Other wafer substrates are available for GaN HEMT fabrication, such as sapphire. The GaN layer has the advantage of wide bandgap properties that facilitate high electron mobility and contribute to the sensor's sensitivity and response time. Within this GaN layer, a two-dimensional electron gas (2DEG) channel forms naturally at the interface with the AlGaN layer. The 2DEG is a thin layer of mobile electrons that is highly sensitive to changes in electric fields and charge density.

The Aluminum Gallium Nitride (AlGaN) layer works in conjunction with the underlying GaN to create the 2DEG channel. The material properties of AlGaN, including its adjustable bandgap and electron mobility, can be finely tuned during manufacturing to optimize the sensor's performance for specific requirements.

The detection area is formed at the surface of AlGaN layer. This area is where capture molecules are immobilized. The capture molecules are designed to selectively bind to specific analytes, initiating a change in the electrical properties of the 2DEG below, which can then be measured and translated into a detectable signal indicating the presence of the target substances.

Directly above the detection area, a detection well is provided. This well is where the sample containing potential target molecule will interact with the capture molecules.

The GaN HEMT sensor can be improved through the systematic optimization of features formed at the wafer level balanced with materials and processes in the fabrication of the functionalized sensor devices of the die attached to the biosensor card connected with the reader electronics PCB. For example, thinning the AlGaN layer in a GaN HEMT sensor has the potential to increase the sensor's sensitivity, as it brings the detection area—where the capture molecule/target molecule binding occurs-closer to the 2DEG. The proximity can enhance the perturbation effect of the bound molecules on the 2DEG, potentially leading to a stronger modulation of the channel's conductivity when a target molecule binds to a capture molecule. This closer interaction means that even small changes at the surface can have a more significant impact on the 2DEG channel, potentially leading to improved sensitivity of the biosensor. The thickness of the AlGaN layer in a GaN HEMT (High Electron Mobility Transistor) device is an important design parameter. In the context of biosensing applications, there's a trade-off to consider: a thinner AlGaN layer can indeed bring the detection area closer to the 2DEG, potentially increasing the sensor's sensitivity, but it can also introduce several challenges.

For example, the AlGaN layer typically ranges from a few nanometers to tens of nanometers. The optimal thickness is often a result of empirical optimization and depends on the specific application. For biosensing, it might be thinned to just above the critical thickness that prevents the introduction of dislocations and other crystal defects.

A thinner AlGaN layer can alter the electrical properties of the HEMT structure. It can affect the 2DEG density and the device's threshold voltage. These properties should remain within certain limits to maintain the device's operational integrity, so part of the systematic improvement of the bare die structure can include a design of experiments aimed at the optimization of the AlGaN layer, and may include an insulator stack comprising different materials that provide the best balance of fabrication costs, biosensor sensitivity and device robustness to withstand all the fabrication processes, shelf life considerations and test performance.

For example, making the AlGaN layer too thin could compromise the structural integrity of the device, potentially leading to mechanical instability or breakdown under stress or during handling. Thinning the AlGaN layer could also increase the likelihood of leakage currents, especially if the layer becomes too thin to effectively confine the electrons in the 2DEG. This could lead to increased noise and decreased signal-to-noise ratio, adversely affecting sensor sensitivity. The thinner the layer, the more difficult it can be to manufacture it with consistent quality and across large wafers. Non-uniformities in thickness can result in device-to-device variability, affecting yield and performance. If the AlGaN layer is made too thin, direct tunneling may occur, where charge carriers tunnel through the AlGaN barrier instead of being properly controlled by the gate. This could severely degrade device performance.

In general, the design and fabrication of GaN HEMT devices will involve a balancing act where the AlGaN layer is made as thin as possible to enhance sensitivity, but not so thin that it induces significant leakage currents, decreases electron mobility, or adversely impacts the 2DEG characteristics.

FIG. 8 illustrates the fully assembled biosensor card as viewed from both the top and bottom perspectives.

The top view photo shows the liquid detection features formed from the top conductive traces. The liquid detection features are useful for determining the flow of the liquid sample just before and after the sample passes over the biosensor.

In the bottom view, the bare die semiconductor sensor is shown mechanically fixed on the biosensor card and electrically connected to the bottom conductive traces via a z-axis conductive adhesive. This anisotropic adhesive material provides a reliable bond that maintains the electrical integrity of the connection while allowing for electrical conduction only in the vertical (z-axis).

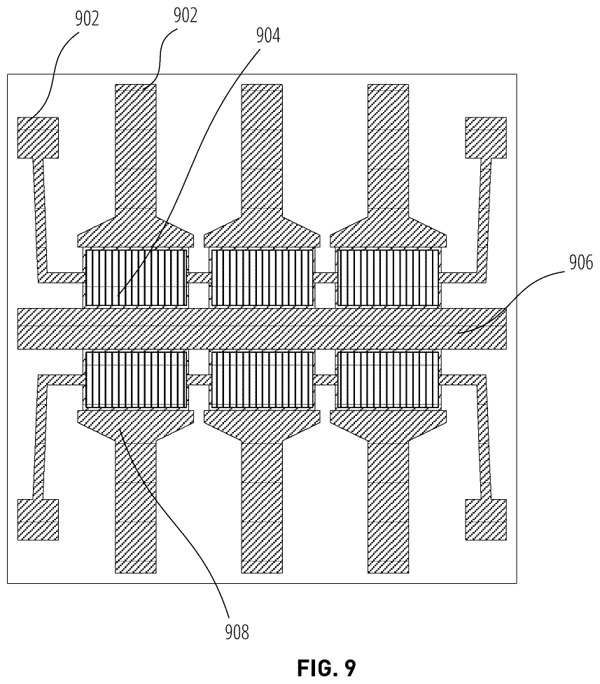

FIG. 9 shows an alternative design of a semiconductor biosensor die with an array of six Gallium Nitride (GaN) High Electron Mobility Transistor (HEMT) devices. In this embodiment, the gate and drain are shared features with individual addressability of each sensor device provided through the individual connection to each source.

An exemplary embodiment comprises a bond pad 902, a detection area 904, a drain 906, and a source 908. That is, the sensor devices each have its own source electrode, while sharing a common gate and drain. This configuration that facilitates the parallel and/or serial readout of test results for multiple biomarkers. This shared structure also reduces the complexity of the biosensor card and enhances the die's compactness, an important consideration since die cost is typically relatable to die size.

Each source bond pad connects the individual sensor devices, and is linked to a separate detection area for each sensor device. These separately addressable detection areas are functionalized with unique capture molecules to enable the detection of various biomarkers, or the same capture molecule can be provided and an average reading from each addressable sensor device taken as the test output. This multi-sensor approach allows for a broad spectrum of diagnostic capabilities, such as simultaneous testing for different viral proteins or pathogens.

In application, this sensor array can effectively analyze complex biological samples, identifying the presence of multiple biomarkers with high specificity and sensitivity. For instance, each sensor device could be functionalized to detect distinct biomarkers associated with various diseases or conditions, offering a comprehensive diagnostic tool within a single semiconductor die.

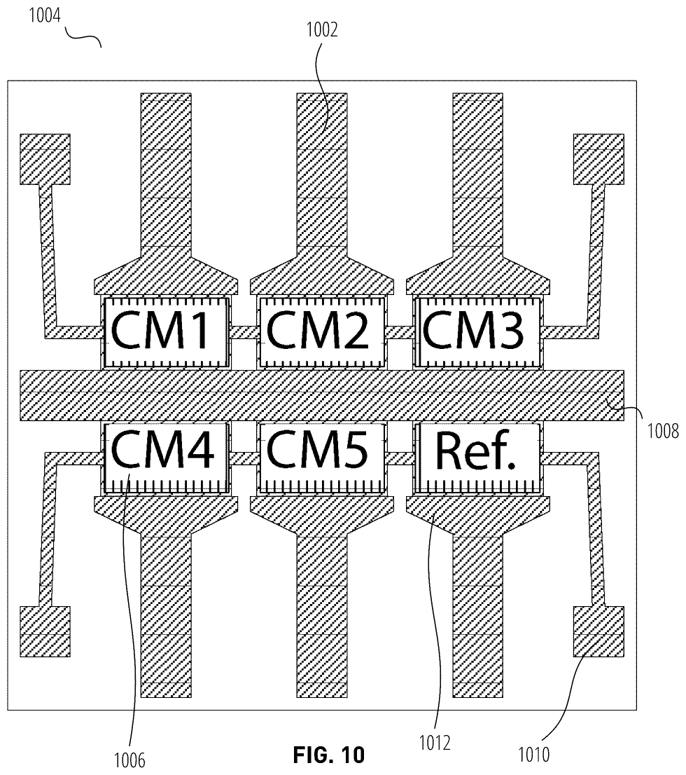

FIG. 10 shows a multi-sensor biosensor die with five test sensors and one reference sensor, all integrated onto a single bare die. Each test sensor device can be uniquely functionalized to detect a specific biomarker. An exemplary embodiment comprises a bond pad 1002, a die 1004, a detection area 1006, a drain 1008, a gate 1010, and a source 1012.

The test sensor devices, labeled CM1 (Capture Molecule 1) through CM5, are functionalized with distinct capture molecules that are selected for their high affinity to particular biomarkers, allowing the sensors to identify and measure the presence of multiple analytes such as the SARS-COV-2 N and S proteins, Flu A and B antigens, and the Respiratory Syncytial Virus (RSV).

The reference sensor device, labeled ‘Ref.’ can be used to normalize the test readings and account for environmental variables like temperature and humidity that could affect sensor performance. It is functionalized with a capture molecule that exhibits representative electrical characteristics similar to the other sensors. This feature enables the reference sensor to act as a control point, maintaining the reliability of the biosensor's output by providing a consistent baseline for comparison.

In practice, a comparator circuit may be used to contrast the signal from the reference sensor with that of each test sensor. This comparison helps to ensure that any signal variations are attributable to the presence of the target biomarker, rather than extraneous environmental factors.

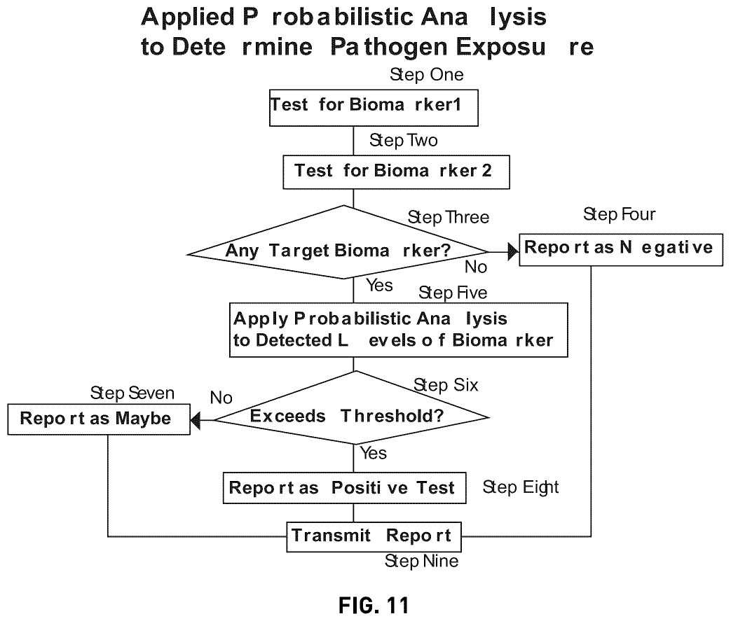

FIG. 11 is a logic flow diagram for Applied Probabilistic Analysis to determine the detection of a target biomarker, and illustrates the operation of an exemplary method, a result of execution of computer program instructions embodied on a computer readable memory, functions performed by logic implemented in hardware, and/or interconnected means for performing functions in accordance with exemplary embodiments.

As an example of a use-case for diagnosing and monitoring a pathogen infection, such as Covid-19, applied probabilistic analysis can be used to improve the predictive model of an individual's infection status and in the aggregate, help to refine the testing results thresholds for an objective quantitative or qualitative testing system. In accordance with an exemplary embodiment, a method is provided for the applied probabilistic analysis to the test results for two or more biomarkers to determine pathogen exposure. Host-generated biomarkers resulting from the immune response of the patient can be combined with virus biomarkers such N- or S-proteins of the SARS-COV-2 virus. In accordance with this example, Biomarker1 is first tested for (step one), Biomarker2 is then tested for (step two). Additionally, BiomarkerN is tested for where N can be any number of multiple biomarkers tested using the inventive testing system. If no target biomarker is detected (step three) then a Negative Test report is generated (step four). If any target biomarker is detected (step three) then probabilistic analysis may be performed depending simply on the detected presence (yes/no) or quantitative analysis (e.g., concentration) of the one or more detected biomarkers (step five). The probabilistic analysis can be performed using an updated probability model where probabilistic multipliers for the tested—for biomarkers are determined for a population. As an example, if a virus outbreak occurs earlier in time in a region or country different from the location of the currently applied testing, the probabilistic multipliers for the tested—for biomarkers can be determined from confirmed cases occurring during the earlier outbreak. A threshold can be determined for the results of the probabilistic analysis based on the probabilistic multipliers obtained from the confirmed cases and help to improve the accuracy of the testing system. As an example, in an electronic biosensor, a threshold voltage for considering a test result as positive can be adjusted based on the probabilistic analysis of previously tested and confirmed positive cases. Over time, the accuracy and confidence of positive and negative determinations is improved based on the history of confirmed cases and obtained threshold voltages. As the database of tested cases grows, the overall testing regimen with interconnected communication, sharing and analysis of tests results is used to automatically improve the accuracy and confidence of future tests.

If the probabilistic analysis does not exceed a threshold (step six) (e.g., low concentration of a particular target biomarker, or the presence of just one weak biomarker indicating likely infection), then a May be Test report is generated (step seven). The ability to detect a possible infection that isn't necessarily confirmed positive could be important during an early stage of a new pandemic or regional outbreak since it is important to identify possible infections and remove the possibly infected individuals from unnecessary contact with other until their infection status can be confirm. If the probabilistic analysis does exceed a threshold (step six) (e.g., high concentration of a particular target biomarker, or the presence of two or more biomarkers indicating likely infection), then a Positive Test report is generated (step eight). The Test Report is then transmitted (step nine) (e.g., in a manner described herein or other suitable transmission mechanism including verbal, digital, written or other communication transmission that adds to the accumulated database of test results).

The logic flow is implemented by a non-limiting embodiment of an apparatus, comprising at least one Processor; and at least one Memory including computer program code, the at least one Memory and the computer program code configured to, with the at least one Processor, cause the apparatus to perform at least the following: detecting one or more biometric parameters using a droplet harvesting structure for converting breath vapor to a fluid droplet for forming a fluid sample and a testing system having a biomarker testing zone for receiving the fluid sample and detecting the biometric parameter, where the biometric parameters are biomarkers dependent on at least one physiological change to a patient in response to a concerning condition such as a virus infection; receiving the one or more biometric parameters and applying probabilistic analysis to determine if at least one physiological change threshold has been exceeded dependent on the probabilistic analysis of the one or more biometric parameters; and activating an action depending on the determined exceeded said at least one physiological change.

In accordance with an embodiment, a biosensor testing device is provided having one or more biometric detectors each for detecting biomarkers as one or more biometric parameters. The biometric parameters are dependent on at least one physiological change to a patient or test subject, such as the production of immune response chemicals, the presence in the body of an active or deactivated virus or virus component, antibodies, antigens, virus RNA, or other biomarker inducing change (including an immune response or viral load count). A microprocessor receives the one or more biometric parameters and determines if at least one physiological change threshold has been exceeded depending on the one or more biometric parameters. An activation circuit activates an action depending on the determined physiological change. The action includes at least one of transmitting an alert, modifying a therapeutic treatment, and transmitting data dependent on at least one physiological change, the one or more biometric parameters, and therapeutic treatment.

For example, a mask-based diagnostic platform may utilize the components described herein, and can also be used to monitor the progression of a disease in a patient, for example, a hospitalized patient that is going through the disease progression of Covid-19, recovering for a heart attack, organ injury, cancer, etc. The at least one physiological change can also be in response to an applied therapeutic treatment that causes a change in the condition of the patient to enable the monitoring of the body's response to an applied therapeutic. The action can include transmitting an alert, modifying a therapeutic treatment, and transmitting data dependent on at least one of the at least one physiological change, the one or more biometric parameters, and therapeutic treatment. The microprocessor can analyze the one or more biometric parameters using probabilistic analysis comprising determining from a data set of the one or more biometric parameters whether the data set is acceptable for deciding that the at least one physiological change threshold has been exceeded. The probabilistic analysis can further comprise applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters.

The MDB system can utilize the logic flow diagram for Applied Probabilistic Analysis in conjunction with an AI-agent that can perform the analysis of the collected biomarker data. The AI-agent can be designed to analyze the aggregated data from the remote server to identify patterns and trends in the data, and determine if a threshold has been exceeded for the at least one physiological change. The AI-agent can also determine if any target biomarker has been detected and perform the probabilistic analysis using an updated probability model.

In addition, the AI-agent can apply a statistical weighting to each of the biometric parameters, which can help to determine the ranking of importance in detecting each of the at least one physiological change for said each of the one or more biometric parameters relative to others of the one or more biometric parameters. This can further help to improve the accuracy and confidence of positive and negative determinations.

The AI-agent can provide feedback based on the identified patterns and trends in the data and the results of the probabilistic analysis. This feedback can be used to improve the efficacy of the remote patient monitoring system by implementing the feedback to optimize at least one hardware, software, and networking component of the remote patient monitoring system.

There are several AI algorithms that can be utilized to automate the analysis of collected biomarker data in accordance with the Applied Probabilistic Analysis process described by the flowchart. One such algorithm is the Bayesian network, which is a probabilistic graphical model that represents a set of variables and their conditional dependencies using a directed acyclic graph. Bayesian networks can be used to model the probability distribution over the biomarkers and other variables, allowing for the calculation of conditional probabilities and the updating of the probability distribution as new data is collected. Other AI algorithms that can be used for biomarker data analysis include support vector machines (SVMs), random forests, and deep learning neural networks.

FIG. 11 shows the detailed procedure for attaching a bare die semiconductor sensor to a support member during the fabrication of the biosensor card. The bare die is attached to the support member so that the detection window in the support member aligns with the detection areas on the sensor devices. An exemplary embodiment comprises a mask 1202, a top surface 1204, a bond pad 1206, a detection area 1208, a z-axis conductive adhesive 1210, a non-conductive adhesive 1212, a liquid detection window 1214, and a support member 1216.

In the die attach operation, a z-axis conductive epoxy bead is applied around the edges of the bare die. The conductive epoxy serves multiple purposes: it secures the bare die to the support member; provides electrical connectivity between the die and the conductive traces on the support member; and seals the sample well defined the by the top surface of the bare die and the walls of the detection window in the support member. The sample well can include additional layers, such a patterned plastic or an external well structure to hold a volume of a liquid sample as a pool in contact with the detection well. As an alternative or in addition to the z-axis conductive adhesive sealing the sample well, a barrier bead of a non-conductive adhesive can be provided that prevents the z-axis conductive epoxy from being exposed to solvents and other materials or processes that could degrade the z-axis conductive adhesive. This non-conductive adhesive may be beneficial depending on the post-die attach processing of the biosensor card. For example, if a solvent used to clean the detection area prior to functionalization would dissolve the cured z-axis conductive adhesive, the non-conductive adhesive could be provided as a barrier to prevent exposure of the z-axis conductive adhesive to the solvent. As another barrier, a gasket may be pick and placed and held in place by the z-axis conductive adhesive bond or the gasket may have a better bond strength to give more flexibility to the choice of z-axis conductive material.

Following the application of the conductive epoxy in the bare die window, the bare die is placed onto the epoxy bead and the epoxy is cured. Pressure can be applied during curing to ensure good physical contact and the formation of a robust electrical and mechanical bond between the die and the support member.

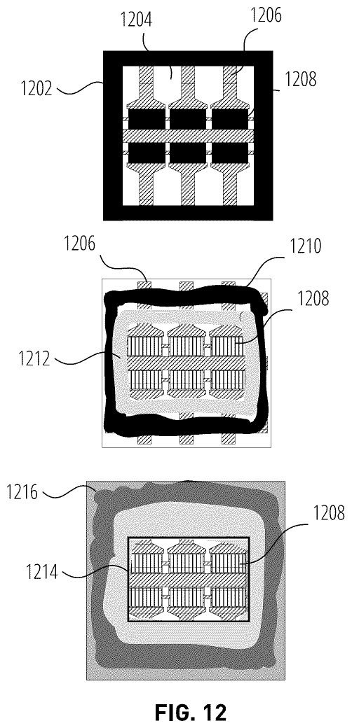

Once the epoxy has cured, a protection layer can also applied on the sensor, leaving the detection areas and bond pads open. This protection layer can be formed from cooperating hydrophobic and hydrophilic structures. For example, at the wafer level or in materials adjacent to the die (e.g., on the biosensor card). During the die attach process, the bare die is placed onto the support member so that the detection window in the support member aligns with the detection area of the sensor devices. The views shown in FIG. 12 are looking from the bottom up, in the actual die attach process the support member is held on a work holder and z-axis conductive epoxy bead is formed at the bare die window of the support member, then the bare die is pick and placed onto the z-axis conductive epoxy bead and pressure applied to ensure a good electrical contact, mechanical attachment and seal. Depending on the type of z-axis conductive epoxy, the applied pressure may be held during a snap or UV curing process.

For example, the z-axis conductive adhesive comprises at least one of a anisotropically conductive epoxy, anisotropically conductive glue, and anisotropically conductive pressure sensitive adhesive film. The conductive adhesive provides at least one of an electrical connection and mechanical attachment, and the respective bond pad and corresponding conductive trace is electrically connected by the z-axis conductive adhesive without causing short circuits between adjacent or other bond pads and conductive traces. The sealing member can be a silicone adhesive or sealing material that forms a barrier between the sample well and the z-axis conductive adhesive.

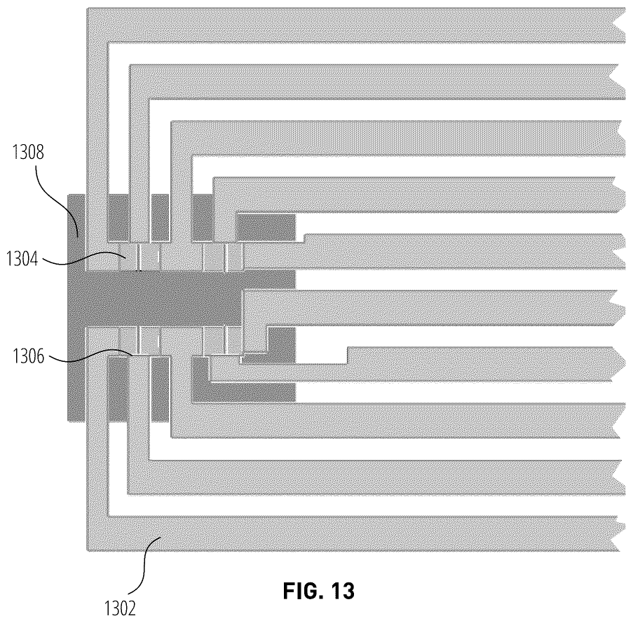

FIG. 13 shows an isolated view of the packaged semiconductor sensor where a bare die semiconductor sensor device is attached to an flex circuit biosensor card using a z-axis conductive adhesive. The view shows an isolated and partial assembly of the biosensor card showing the arrangement of conductive traces, detection areas, sample wells, and other relevant structural features of a biosensor card assembly with four individually addressable and functionalizable sensor devices on a single bare die. The exemplary embodiment comprises a conductive trace 1302, a detection area 1304, a detection window 1306, and a die 1308.

Conductive traces are patterned on the substrate of the biosensor card to provide electrical pathways to enable signal detection and transmission from the sensor elements to a data processing unit, e.g., reader electronics provided on a printed circuit board. Individually addressable detection areas and individually accessible sample wells are provided so that a fluid sample can be tested for different target analytes by each of the four individually addressable sensor devices.

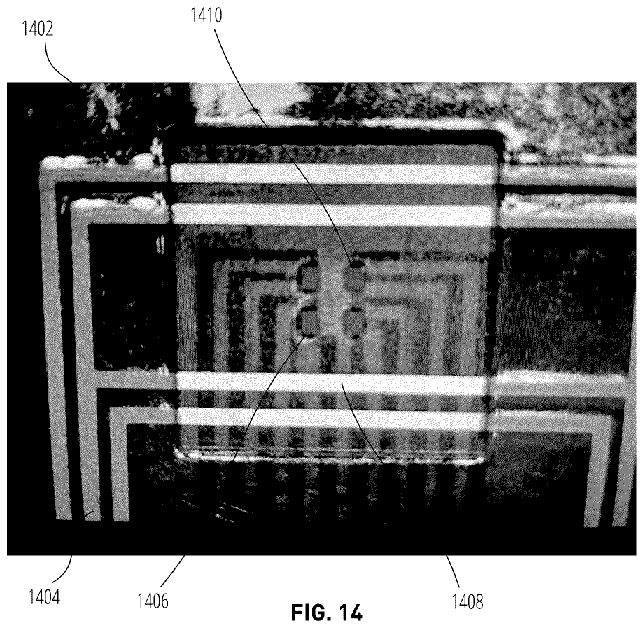

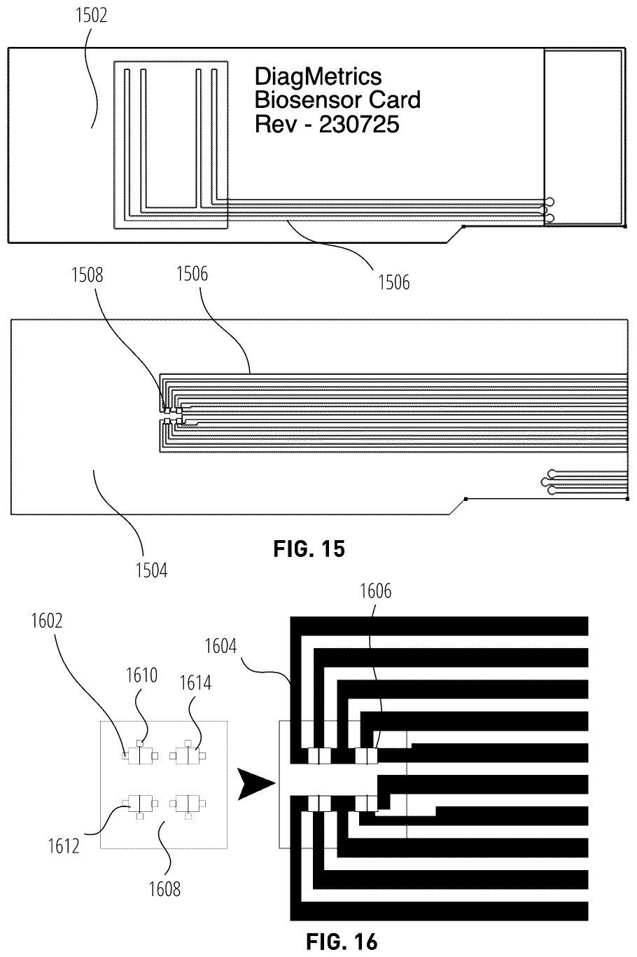

FIG. 14 shows an embodiment of a completed biosensor card with four individually addressable sensor devices. Each sensor is equipped with its own sample well, allowing for the detection of distinct target molecule from the same fluid sample. The detection areas of each of the sensor devices can be individually functionalized to perform multiplexed testing within a compact and integrated system. This configuration allows for simultaneous and selective analysis of various biomarkers in the same fluid sample.

The system comprises a biosensor card 1402, a conductive trace 1404, a detection window 1406, a liquid detection features 1408, and a sample well 1410.

The completed biosensor card is a multi-sensor platform where each sensor device has an individually assessable sample well. The detection windows, visible in the center of the photo, receive the fluid sample. The biosensor card includes liquid detection features that are used to determine the flow of the liquid sample before and after the detection areas.

This biosensor card allows for the simultaneous analysis of different target molecule within a single fluid sample by using four independently addressable sensor devices. Each of these sensor devices can be individually functionalized to detect specific biomarkers enabling multiplexed testing capabilities for scenarios where multiple assays need to be performed concurrently, such as in comprehensive medical diagnostics or complex environmental analyses.

The biosensor card assembly includes a bare die semiconductor sensor with a top surface including two or more bond pads and at least one detection area. A support member has at least a corresponding number of conductive traces as the bond pads on the bare die. The conductive traces are provided on at least a bottom side of the support member for connecting with the bond pads of the bare die. The support member has a through-hole detection window aligning with the detection area of the bare die. A conductive adhesive is provided between each bond pad of the bare die and a corresponding conductive trace of the support member. The conductive epoxy provides an electrical connection between a respective bond pad and a corresponding conductive trace.

FIG. 14 presents a detailed schematic of a biosensor card, delineating various integral components and their arrangement for multiplexed biomolecular detection. The card comprises a top side and a bottom side, ensuring structural integrity and facilitating electrical connections via conductive traces. Central to the design is the detection window, which is aligned with individually accessible sample wells situated on the top side of the biosensor card.

An embodiment comprises a biosensor card 1502, a bottom side 1504, a conductive trace 1506, and a detection window 1508.

Each sensor device is independently addressable, enabling the biosensor card to analyze distinct target molecule concurrently within a singular fluid sample. This capacity for individual functionalization of the sensor devices allows for a comprehensive analysis of multiple biomarkers.

The support member can be fabricated from a flexible or rigid substrate material and the conductive traces are formed on the flexible substrate through at least one of an additive manufacturing process and a subtractive manufacturing process.

FIG. 16 shows an exemplary layout of a biosensor card with conductive traces leading to distinct sensor devices. The exemplary embodiment comprises a bond pad 1602, a conductive trace 1604, a detection window 1606, a die 1608, a gate 1610, a detection area 1612, and one or more semiconductor sensor devices 1614.

This layout enables multiplexing testing where different sensor devices on the same biosensor card can be selectively activated and read, allowing for complex diagnostic assays to be performed in parallel.

The addressable detection wells are arranged to correspond with the detection areas, and each well is configured for analyzing specific target molecule from a fluid sample introduced through the detection window. The conductive traces are patterned for independent electrical addressing of each sensor device, enabling the simultaneous and separate analysis of multiple biomarkers.

This layout enables multiplexing capability, where different sensor areas on the same card can be selectively activated and read, allowing for complex diagnostic assays to be performed in parallel. Such a configuration is key for high-throughput screening and real-time monitoring of diverse biological samples, indicating the card's potential utility in advanced medical diagnostics, environmental sensing, and bioanalytical systems.

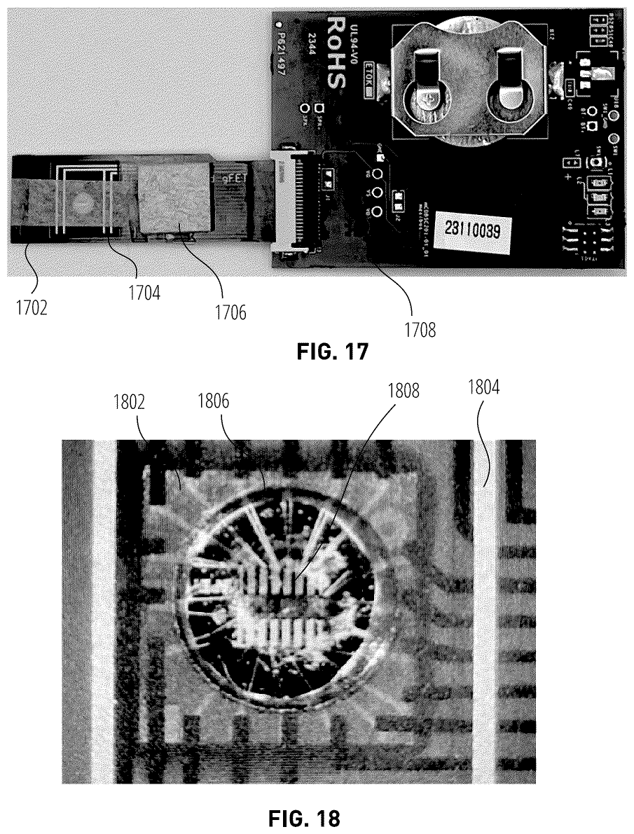

FIG. 17 shows an embodiment of the biosensor card interfaced with a printed circuit board (PCB) through a zero insertion force (ZIF) connector. The biosensor card include microfluidic channels comprising a filter paper, guiding a fluid sample along a defined path. Initially, the sample encounters a liquid detection feature, which could be an assay or sensor that preliminarily assesses the presence or characteristics of the sample.

The sample then flows over the detection wells where the sample interacts with the detection area of the biosensor. As the sample exits the detection wells, it passes over a second detection feature. This sequential flow ensures that the sample is analyzed both before and after the primary detection event.

The compressed cellulose sponge adjacent to the microfluidic channels serves as a reservoir or wicking material to facilitate the capillary action that drives the sample through the microfluidic system without the need for external pumps.

The PCB, which the biosensor card connects to via the ZIF connector, provide electronic components to transduce the sensor signals into readable data. The support member is designed to facilitate quick-release engagement with a data processing unit via a mechanical and electrical connector interface, allowing for rapid interchangeability of the biosensor card.

FIG. 18 showcases a close-up view of a portion of a biosensor card, specifically focusing on the components for sample detection. Central to the image is a detection window, a transparent or open area that allows a fluid sample to interact with the sensor's detection area. The detection area, typically functionalized with specific reagents or biological elements, is the active site where the target molecule within the sample are captured or analyzed.

Surrounding the detection area is the detection well, which is likely designed to contain the fluid sample and ensure that it remains over the detection area for a sufficient period, enhancing the sensor's ability to detect the target molecule present.

The arrangement suggests that the detection window facilitates the introduction of the sample, the detection well serves to confine the sample over the detection area for analysis, and the detection area itself contains the elements for the biochemical interaction or reaction that leads to the detection of specific biomolecules. This setup is integral for various applications, including medical diagnostics, environmental monitoring, or any field requiring precise molecular detection.

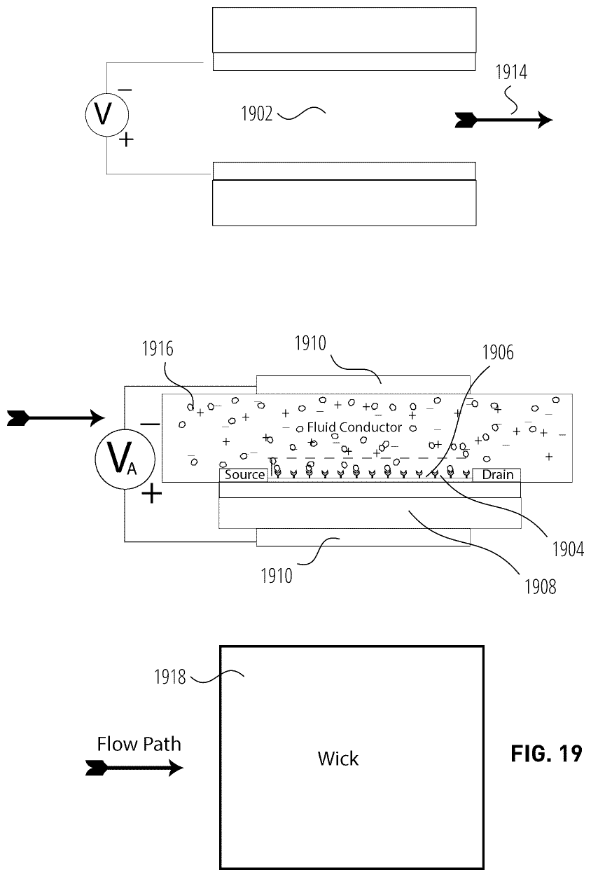

FIG. 19 shows an electrostatic control system for use with a semiconductor sensor to enhance the detection and analysis of target molecule in a fluid sample. Driving electrodes create a controlled electrostatic field across the sample flow path. The electrostatic field modulates the motion and orientation of charged particles and polar molecules within the sample. The electrostatic field can be controlled at various locations along the sample flow path so that when the sample flow passes over the detection area, the target molecule are better positioned for binding with the immobilized capture molecules. An exemplary embodiment comprises an accumulator 1902, capture molecules 1904, a detection area 1906, a die 1908, driving electrodes 1210, a flow path 1914, and a target molecule 1916.

An integrated biosensor card and bare die sensor assembly is provided for targeted biomarker detection. A semiconductor die has a top surface with a least one sensor device and at least one sensor area and bond pads associated with each sensor device. As shown in other Figures, a support member having a bottom side with conductive traces corresponding to the bond pads supports the semiconductor die and connects the semiconductor devices of the die to a printed circuit board. An accumulator in fluid communication with the sensor areas applies an electrostatic field to a fluid sample for aligning target biomarkers within a fluid sample. The accumulator can be provided on the biosensor card and in the flow path of the liquid sample, and facilitates enhanced detection by modulating the orientation and proximity of target biomarkers to the immobilized capture molecules at the sensor areas.

A semiconductor sensor, such as a graphene field effect transistor sensor, has optimized sensitivity that is related to the Debye screening length and the distance and orientation of the capture molecule binding sites from the detection surface. To facilitate the binding interactions, an accumulator can be provided in the flow path of the liquid sample before the sample reaches the detection area. An electrostatic field selectively concentrates and orients the target molecules. The signal applied to the conductor of the accumulator can be controlled so that the momentum of the polar molecules is altered to favor the movement of the binding site of the target molecule towards the capture molecules immobilized at the detection area.

The electrostatic field selectively controls the orientation and movement of target molecule and concentrate them in the direction of the capture molecules immobilized on the detection area. The applied electrostatic driving force induces rotational movements in polar molecules, positioning them for binding and contributing the captured charges at that detection surface to influence the flow of electrons as a detected signal. By orienting the molecules correctly, the binding efficacy to these sites is increased, leading to enhanced sensitivity and specificity of the sensor. Similarly, the immobilized capture molecule can also be a polar molecule. The electrostatic field orients the capture molecule and the applied signal can be control so that the orientation is optimized for the binding interaction with the target molecule.

The flow path can include hydrophobic and hydrophilic structures formed at the wafer level that control the flow and volume of the fluid sample to improve the wetting of the detection area and form a pool of the flowing liquid sample.

This flow path layer can be formed from cooperating hydrophobic and hydrophilic structures to control the direction and rate of the liquid sample flow. For example, at the wafer level or in materials adjacent to the die (e.g., on the biosensor card), microfabricated, screen printed or otherwise formed or applied pattern of surface energy features can be made. An accumulator applies and electrostatic field to the fluid sample passing between two insulated conductors. The electrostatic field can be applied in pulse or patterns to give momentum to a polar molecule to cause rotation. For example, an on/off DC (direct current) pulse can be tuned to maximize the separation of the polar target molecule from the other ions and polar constituents in the sample.

Creating hydrophobic and hydrophilic patterns at the wafer level enables the control of the sample fluid flow using surface modifications that alter the wetting properties of specific areas on the die. These surface properties are can be controlled at the microscale for directing and confining fluid samples to targeted areas, such as the detection zones in a biosensor. The hydrophobic and hydrophilic patterns can be fabricated by selectively applying these monolayers through stencil printing or microcontact printing. Plasma treatments can also be used to modify surface energy. Exposing the detection surface to an oxygen plasma can make it more hydrophilic, while a fluorocarbon plasma can make it more hydrophobic.

The hydrophilic/hydrophobic patterns can converge the liquid sample flow path to the detection area and a pattern of more and less hydrophobic/hydrophobic regions on the detection area can control the density of capture molecules immobilized during the functionalization process. The pattern can be made to facilitate wetting active regions of the detection area. Hydrophobic boundaries can also be designed around the detection area to create a microwell or pool where the sample can accumulate. A fine grid of hydrophilic boundaries on the detection area can also control both the density and uniformity of the immobilized capture molecules, as well as utilize the surface tension of the fluid sample to draw the sample into contact with hydrophobic detection area active regions bounded by the hydrophilic grid. Hydrophobic boundaries can control the size of the pool to determine the volume of the sample that interacts with the sensor to enable quantitative analysis. In general, standardized wetting and pooling can lead to more consistent sample volumes and sensor interactions, improving the reproducibility of the sensor's readings.

The accumulator is provided in fluid communication with the sensor areas, the accumulator applies an electrostatic field to the fluid sample for aligning target biomarkers within a fluid sample. The accumulator facilitates enhanced detection by modulating the orientation and proximity of target biomarkers to the sensor areas.

For a Gallium Nitride High Electron Mobility Transistor (GaN HEMT) biosensor configuration, the general principles described for the g-FET remain consistent. However, the material properties and operation of the GaN devices can be leveraged to further refine the system. GaN has a high electron mobility and a wide bandgap, which is beneficial for biosensing applications. GaN surfaces can be modified with hydrophobic and hydrophilic patterns to control fluid flow much easier than forming patterns of graphene at the microscale. The robustness of GaN allows for a variety of surface treatments that can create these patterns without compromising the integrity of the sensor. The functionalization of the GaN detection area to immobilize capture molecules can benefit from hydrophilic/hydrophobic patterning. For instance, a hydrophilic grid can attract the sample and promote even distribution across the active area, while hydrophobic regions can prevent non-specific binding, improving signal-to-noise ratios in the sensor output.

The fine-tuning of pool sizes via hydrophobic boundaries around GaN detection areas can facilitate precise quantitative analysis by controlling the volume of the sample interacting with the sensor. GaN's stability under various environmental conditions allows for such features to be implemented with high reproducibility. GaN technology is compatible with standard semiconductor microfabrication techniques. Microfabricated patterns for controlling sample flow at the wafer level can be implemented on GaN devices using conventional etching, photolithography, or newer methods like direct laser writing.

At least one of hydrophilic and hydrophobic patterns are formed on at least one of the top side of the biosensor card and the top surface of the bare die control flow and positioning of the fluid sample over the detection area. The hydrophilic and hydrophobic patterns are arranged to create microchannels that direct the fluid sample towards the detection area. The hydrophobic patterns are located around a periphery of the detection area to contain the fluid sample. A microstructured surface on the detection area can includes a combination of hydrophilic and hydrophobic regions designed to modulate sample volume and fluid dynamics for optimizing surface wetting properties of the detection area.

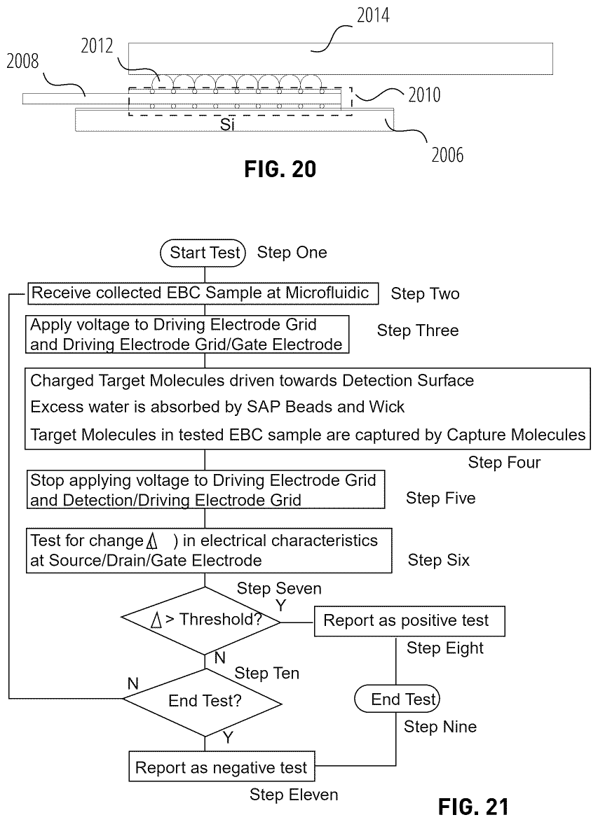

FIG. 20 is a side view of a sensor for detecting target molecules in a fluid sample with a detection area for receiving a fluid sample and a top driving electrode and a bottom driving electrode where an electric potential drives the target molecule towards the capture molecules to concentrate the target molecules in a portion of the fluid sample received at the detection interface. The system includes a sensor device, a fluid transfer mechanism, a detection area, a super absorbent polymer (SAP), and a wick.

The top and the bottom driving electrodes can be disposed in the detection area. A wick absorbs excess fluid from the sample. The fluid transfer mechanism conducts a portion of the fluid sample containing relatively less target molecule through the SAP which selectively absorbs water and ions and leaves behind larger molecules, such as a target protein molecules. The wick absorbs the water and ions from the SAP leaving another portion of the fluid sample containing relatively more target molecule flowing towards the capture molecules at the detection area of the sensor device.

FIG. 21 is a flowchart of the steps for concentrating the target molecule in a fluid sample and testing for a change in electrical characteristics of a functionalized transistor sensor.

In accordance with another non-limiting embodiment, a method for detecting a target molecule from a fluid sample comprises the steps of: receiving the fluid sample comprising the target molecule at a microfluidic channel; transferring the fluid sample from the microfluidic channel to a detection interface of a sensor device, the sensor device comprising a detection area for receiving the fluid sample and having the detection interface functionalized with capture molecules, a top driving electrode and a bottom driving electrode defining a gap there between, and a fluid conductor disposed in the gap for conducting the fluid sample through the gap, wherein an electric potential applied to the top and the bottom driving electrode drives the target molecule towards the capture molecules as shown, for example, in FIG. 19 to concentrate the target molecule in a portion of the fluid sample received at the detection interface. The sensor comprising a field effect transistor having a gate disposed in electrical communication with the detection interface that is functionalized with the capture molecules, and a source and drain on either side of the gate or some other semiconductor device feature arrangement where the conduction of charges is affected by the presence of target molecules in a tested sample (see, for example, FIG. 2). At least a portion of at least one of the top and the bottom electrode can be disposed at or in electrical or electrostatic communication with the detection area. A gate, source or drain electrode of the sensor may comprise, for example, the top driving electrode and the sensor device substrate or other conductive member comprise the bottom driving electrode so that an electrostatic field is applied through the liquid sample that causes molecules and ions to orient and or move in response to the applied electrostatic field. The arrangement of conductors and features described herein are illustrative of possible variations. Various arrangements of the conductors are possible with the aim of establishing a controllable electrical field that causes a change in orientation and location of the molecules and ions present in the sample material being tested. The electrodes and conductive members of the system can be driven with applied electrical signals intermittently and selectively for applying electric potentials for driving the target molecule and for taking a test reading of a change in an electrical characteristic at or between any of the source, drain and gate, or other conductive member of the system.

In an example use, where the system is used to detect a target molecule contained in a liquid exhaled breath condensate (EBC) biosample, at the start of the test (step one) the EBC sample is received at the microfluidics or fluid transfer mechanism of the system (step two). A voltage is applied to a driving electrode grid that supports the SAP beads, or other top and bottom electrode configuration where an electrostatic field is created in the detection interface (step three). The applied voltage drives the polar target molecule and ions in the EBC sample towards and away from the capture molecules at the detection interface of the biosensor depending on the applied polarity and the particular charge distribution of the molecules and ions in the EBC sample. The aim is to concentrate the target molecule in a portion of the fluid sample received at the detection interface, with excess water absorbed by SAP and/or the wick. The target molecule in the tested EBC sample are captured by the capture molecules immobilized at the detection interface causing a detectable change, for example, in the flow of correct from the source to the drain of the sensor device. To test for the detectable change, the electric potential for driving the target molecule is stopped (step five). A test reading taken of a change in an electrical characteristic caused by the captured target molecule affecting the electron charge mobility at the detection interface. Stated otherwise, the electric potential for driving the target molecule can be applied intermittently with taking a test reading of a change in an electrical characteristic at the source/drain/gate electrodes of the transistor biosensor (step six). Alternative electrode and biosensors configurations can also be used, including printed electrodes with nanoparticle, nanotube, metal, semiconductor and/or organic detecting interface materials. If the change in electrical characteristics is greater than a threshold value (step seven) then a positive test is reported (step eight) and the tested ended (step nine). If the change is less than the threshold value (step seven) then it is determined if it is time to end the test (step ten). For example, the test can be ended after a given period of time or a given amount of fluid sample is tested. If it is not time to end the test (step ten) then the process flow returns to receiving more of the collected EBC sample at the microfluidic (step two). If it is time to end the test (step ten) and the change in electrical characteristics has not exceeded the threshold (step seven), then a negative test is reported (step eleven) and the test is ended (step nine).

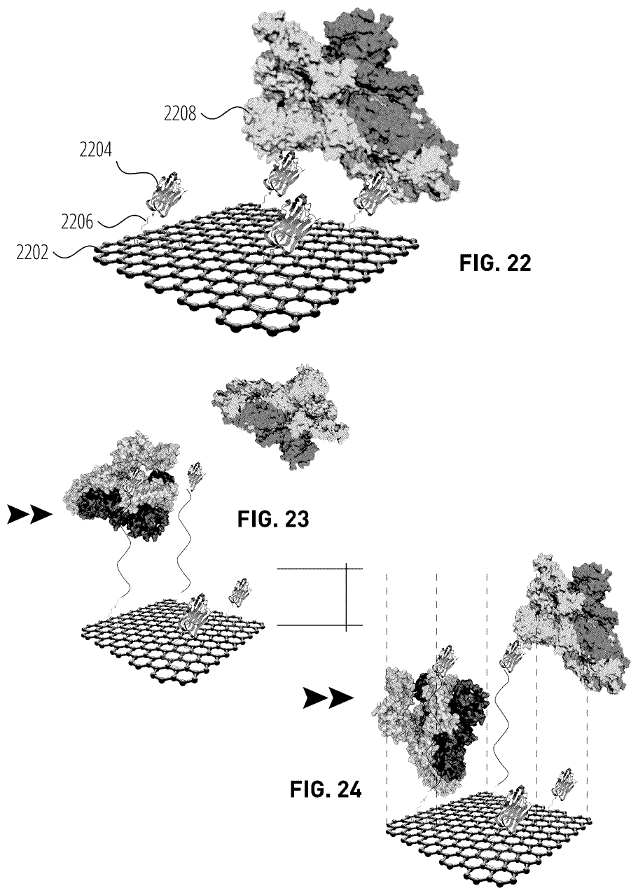

FIG. 22 shows a monolayer of graphene of an electrolyte-gated graphene field-effect transistor. The graphene layer is functionalized with nanoCLAMP capture molecules through a pyrene linker, where two or more nanoCLAMP capture molecules are binding to different binding sites of a target molecule.

As an example, the testing unit provided in a mask-based diagnostic system may comprise a g-FET biosensor having a detection interface comprising a graphene layer functionalized with capture molecules. A mask-based syndromic testing device including sensor devices can be designed to bind to biomarkers of FluA, FluB, SARS N-protein (more conserved, slower to mutate protein across SARS viruses) and S-protein (faster to mutate, main cause of the SARS-COV-2 variants).

Similar to the immobilization of capture molecules on the graphene monolayer detection area shown, a GaN HEMT sensor (or other sensor device) can include the capture molecules immobilized on a detection area that provides a detectable signal when one or more binding events between target and capture molecules occurs.

FIG. 23 illustrates a graphene detection interface with nanoCLAMP capture molecules immobilized on the graphene surface by linker molecules, with a portion of the capture molecules immobilized at a greater distance by a longer link than other capture molecules immobilized on the detection interface with shorter linkers. Also shown is a non-target protein, the target protein, and nanoCLAMPs. The target and non-target molecules are free floating in a liquid sample medium, such as EBC, blood, serum, saliva, urine, sweat, interstitial fluid, tears, sputum, lavage, etc.

FIG. 24 illustrates an electric field potential applied in the detection area that drives a target molecule and non-target molecule towards the capture molecules where the target molecule is captured by a capture molecule extending on linker molecules a relatively longer distance from the detection.

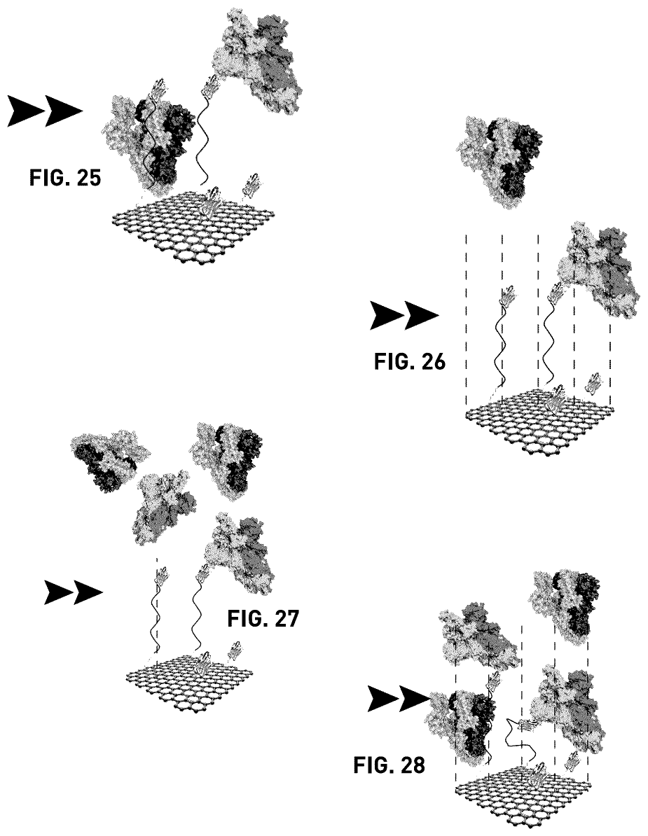

FIG. 25 illustrates the electric field potential removed from detection area. In accordance with a non-limiting embodiment, a portion of the capture molecules is immobilized on the detection interface at a greater distance than another portion immobilized on the detection interface. As a target molecule is driven towards the detection interface, it may encounter a capture molecule that is tethered at a relatively longer stand-off distance and get captured and thus immobilized and then also tethered to the detection interface.

FIG. 26 illustrates an opposite polarity electric field potential that drives the target molecule and non-target molecule away from the detection interface.

The target molecule remains tethered by the capture molecule that is immobilized by the relatively longer linker on the detection interface. The non-target molecules and ions contained in the fluid sample along with the target molecule that are not captured are driven further away from the detection interface by the opposite polarity electric field. The driving circuit reverses polarity of the applied electric potential to cyclically drive non-target molecules from the detection area (making room for another target molecule to migrate towards the detection interface) while target molecules captured by capture molecules immobilized on the detection interface are retained in the detection area. The molecules and ions that are of opposite polarity as the target molecule will be driven away from the capture molecules when the target molecule are driven towards the capture molecule, and vice-versa. The net effect is that the binding opportunity for target and capture molecules increases through the application of the applied electric field and once the binding occurs the target molecules are tethered to the detection detection interface (e.g., graphene surface in the case of a gFET sensor).

FIG. 27 illustrates additional target molecule molecules and non-target molecules flowing into the detection area, or otherwise being brought into proximity with the capture molecules where the applied electric field can selectively tether additional target molecule and increase the detectable signal.

FIG. 28 illustrates the electric field potential applied in the detection area, driving the tethered target molecule into position to be captured by a capture molecule that is immobilized by a relatively shorter linker molecule. Also, another target molecule is shown captured by another capture molecule extending on linker molecules a relatively longer distance from the detection interface.

This cycle of reversing electric field polarity can be used to concentrate the target molecules in a portion of the fluid sample received at the detection interface. Some target molecules may get captured directly at the shorter standoff distance capture molecules, or through a pumping action of cycling the polarity of the applied electric field, the target molecules over time become more prevalent and captured by the capture molecules at the detection interface while the non-target molecules and ions present at the detection interface are reduced. Through the application of the electric field and the tethering and selective binding by the immobilized capture molecules, the resulting different capture molecule standoff distances from the detection surface produce different sensor-to-antigen binding site distances with the capture molecules that bind to the target molecule contributing a stronger signal response of the detection circuit.

This sensor system is designed to enhance molecular detection by utilizing both physical arrangement and electric fields to manipulate and measure molecules within a sample.

The sensor includes a source and a drain on the substrate that create a pathway for charges to flow, which is influenced by the binding events between the capture and target molecules. This change in charge flow is a direct indicator of molecular binding.

To further refine detection, the system employs top and bottom driving electrodes. These electrodes generate an electric field that orients and directs the movement of molecules within the sample, guiding them towards or away from the detection area. This arrangement not only increases the likelihood of target molecules binding with their corresponding capture molecules but also aids in the removal of non-specific ions and molecules. A washing process is incorporated to clear the detection area of these non-specific entities, enhancing the signal from the bound target molecules.

A unique aspect of the system is its ability to adjust the electric field dynamically through a feedback mechanism that responds to real-time data on target molecule binding. This allows for precise control over molecular interactions at the detection site.

The sensor system can also reverse the polarity of the electric field cyclically, effectively repelling non-target molecules and preventing them from interfering with the detection process. This feature is particularly useful in complex samples from sources like exhaled breath condensate, blood, or urine, where numerous non-target molecules may be present.

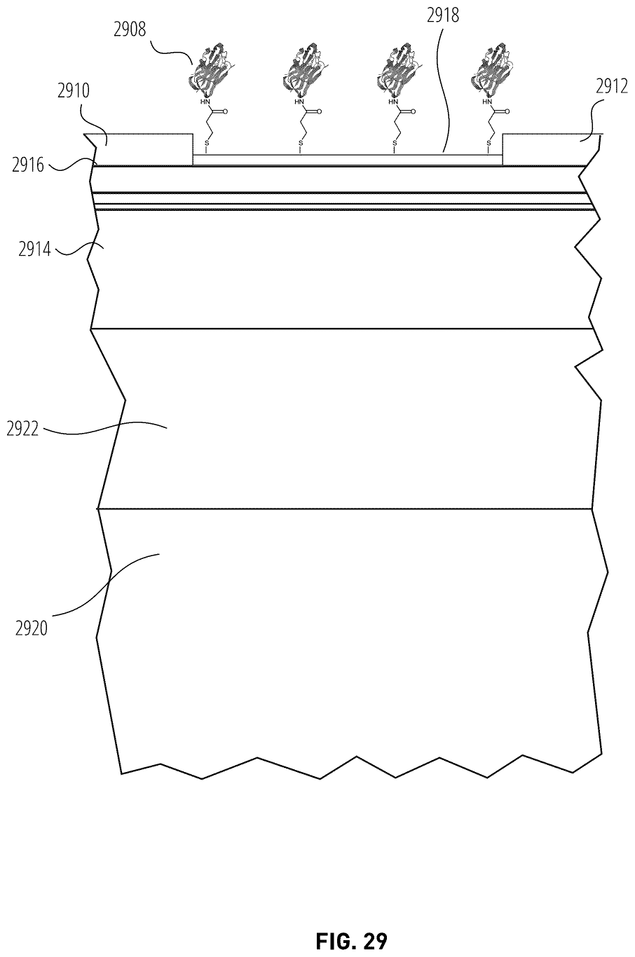

FIG. 29 shows a semiconductor sensor device fixed to a large thermal mass, such as a the EBC Collector, through a conductive adhesive to remove internally generated heat from the sensor during operation. The system comprises a capture molecules 2908, a source 2910, a drain 2912, a substrate 2914, a 2DEG 2916, a detection area 2918, an EBC Collector 2920, and a thermally conductive adhesive 2922.

The Exhaled Breath Condensate (EBC) Collector of a mask-based diagnostic is used to convert exhaled breath vapor into exhaled breath condensate. The EBC Collector provides a large, chilled thermal mass to enhance the performance of the sensor used in a mask-based diagnostic system. For example, phenomenons of the Kink Effect and Trapped Charges can be utilized with the EBC Collector thermal mass to improve sensor functionality.

In semiconductor devices, particularly those involving field-effect transistors (FETs), the Kink Effect refers to a sudden increase in drain current at a certain voltage. This phenomenon typically occurs due to impact ionization and the subsequent trapping of charges at or near the drain region. For a GaN sensor embedded in a diagnostic mask, the Kink Effect could cause non-linear changes in sensor response as the device heats up during operation. Normally, this would be undesirable as it introduces unpredictability in sensor readings. By effectively using the EBC Collector as a heatsink, the temperature rise in the sensor can be minimized. This stable thermal environment helps to suppress the Kink Effect by reducing the chances of impact ionization, which is more pronounced at higher temperatures. Keeping the sensor at a lower and more stable temperature via the chilled EBC Collector can help maintain the integrity of the electrical characteristics of the sensor, leading to more consistent and predictable measurements.

Also, in semiconductor sensors, trapped charges in the dielectric or at the interface can lead to threshold voltage shifts and other instabilities. These effects are usually exacerbated by temperature fluctuations. The large chilled mass of the EBC Collector can help maintain a consistent temperature, thus minimizing the conditions that lead to charge trapping. By keeping the sensor cool, the likelihood of charge mobility that can lead to trapping is reduced, thereby improving the long-term reliability and response accuracy of the sensor.

Leveraging the large chilled mass of the EBC Collector to manage thermal effects and charge dynamics can significantly enhance the performance and reliability of the GaN sensor. This approach not only stabilizes sensor operation but also potentially extends the lifespan and accuracy of the device in capturing and analyzing exhaled breath condensate in medical diagnostics.

In an exemplary configuration, the thermal mass of the EBC Collector is frozen water. The thermal conductivity of ice at 0° C. is approximately 2.2 W/mK. The EBC Collector comprises a thermal mass of frozen water having a thermal conductivity of approximately 2.2 W/mK, which facilitates efficient heat transfer from the semiconductor sensor to the EBC Collector, thereby minimizing thermal gradients and enhancing sensor stability.

While the thermal conductivity of ice (2.2 W/mK) is not as high as metals, it is sufficient to effectively dissipate heat generated by the sensor during operation, especially since the surface of the EBC Collector that the bare die sensor is attached to can be made of a highly thermally conductive material such aluminum or a moderately conductive material such as Teflon. The use of ice as a thermal mass helps to remove heat from the sensor, preventing local overheating and maintaining a more uniform temperature distribution across the sensor. The ability to maintain a stable temperature reduces the likelihood of thermal effects such as the Kink Effect and charge trapping. By keeping the sensor at a consistent temperature, the EBC Collector helps in suppressing impact ionization and/or other effects that would otherwise lead to non-linear changes in the sensor response as the device heats up. This stability can help obtain precision and reliability of the sensor device, and reproducibility when considered wafer to wafer, device to device, test to test.

A stable thermal environment provided by the chilled EBC Collector also minimizes the conditions that lead to charge trapping within the semiconductor material. Charge trapping often results in threshold voltage shifts and can degrade the sensor's performance over time. By maintaining a cooler temperature, the EBC Collector reduces charge mobility, thereby decreasing the likelihood of these detrimental effects.

The semiconductor sensor system described consists of a semiconductor sensor equipped with a source, a drain, and a substrate designed to generate a two-dimensional electron gas (2DEG) below its detection area. This detection area has immobilized capture molecules that are specifically designed to detect particular target molecules. A diagnostic system incorporates an Exhaled Breath Condensate (EBC) Collector, which functions as a chilled thermal mass to condense exhaled breath vapor into exhaled breath condensate. The EBC Collector is directly connected to the semiconductor sensor through a thermally conductive adhesive. This enhances heat transfer from the sensor to the EBC Collector. The stable temperature environment provided by the EBC Collector helps to mitigate thermal effects that typically induce non-linear responses and charge trapping in the sensor.

Notably, the EBC Collector includes a thermal mass made of frozen water, with a thermal conductivity of approximately 2.2 W/mK, although other materials with different thermal conductive can be used for the thermal mass. This configuration is particularly effective in minimizing thermal gradients and improving the stability and reliability of the sensor's readings. The sensor itself can be made from gallium nitride (GaN), which benefits significantly from the thermal management capabilities of the EBC Collector, especially in reducing impact ionization that can occur at higher operating temperatures-commonly referred to as the Kink Effect.

Additionally, the system can be provided with a temperature monitoring system. This temperature monitoring component is either integrated with the sensor or positioned nearby to provide continuous real-time feedback on the sensor's temperature, ensuring the device operates within its optimal temperature range for accurate and reliable performance.

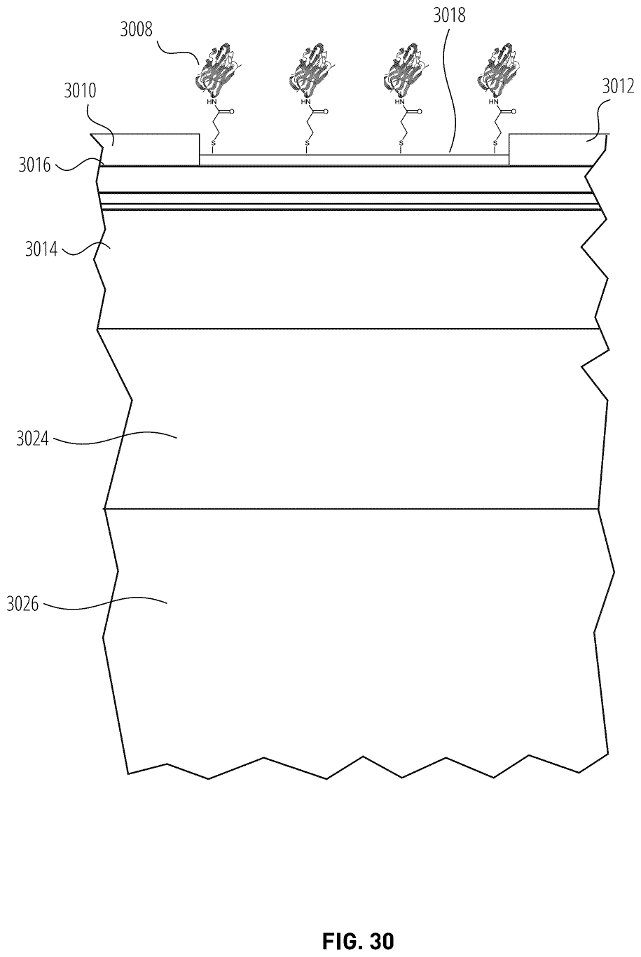

FIG. 30 shows a semiconductor sensor device fixed to an insulator to thermally isolate the sensor during operation. The system comprises a capture molecules 3008, a source 3010, a drain 3012, a substrate 3014, a 2DEG 3016, a detection area 3018, an adhesive 3024, and an insulator 3026.

The sensor configuration shown in FIG. 30 provides a highly stable environment for sensitive measurements where the thermal effects caused by functioning of the sensor are maximized. In this case, instead of sinking away the generated heat (as described above), the internally generated heat is maintained in the sensor bulk by thermally isolating the sensor. The system can include both a Device Under Test (DUT) sensor and a reference sensor, both devices thermally isolated so that the thermal effects during operation are enhanced. The DUT sensor is exposed to a liquid sample containing target molecules, while the reference sensor remains unexposed. This configuration allows for comparative analysis, where the reference sensor serves as a baseline to account for any potential non-target related changes in the sensor's environment or in its own material properties.

The semiconductor sensor system described includes a substrate equipped with a detection area containing capture molecules specifically designed for detecting target molecules. The detection process relies on a two-dimensional electron gas (2DEG) system formed between a source and a drain located on the substrate, which measures electrical properties influenced by molecular interactions within the detection area. To ensure thermal stability during operation and minimize external thermal effects, the sensor incorporates an insulator bonded to the substrate using an adhesive. This insulating setup thermally isolates the sensor, maintaining the operational integrity and accuracy of readings.

Additionally, the sensor system features a dual-sensor setup comprising a Device Under Test (DUT) sensor and a reference sensor. The DUT sensor is exposed to a liquid sample containing target molecules, whereas the reference sensor remains unexposed, serving to reduce thermal variations caused by electrical current flow from the source to the drain. Furthermore, the system integrates a temperature monitoring mechanism that provides real-time feedback on the sensor's temperature, ensuring that it operates within specified limits for reliable performance.

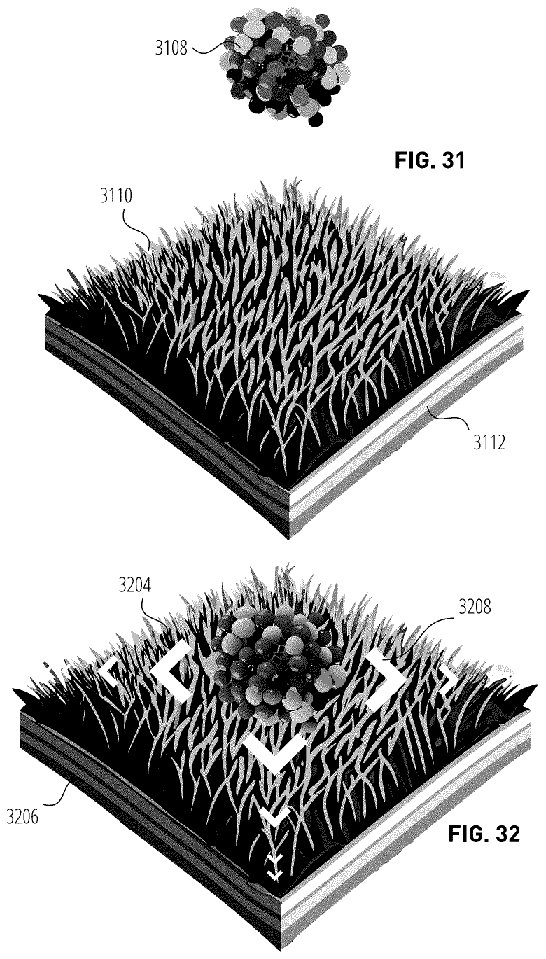

FIG. 31 stylistically illustrates a field of capture molecules immobilized on the detection area of a sensor device, with a single molecule poised to bind with one or more of these capture molecules. The system comprises a target molecule (3108), capture molecules (3110), and a die (3112).

Previous research has described a phenomenon where an electrostatic change from a single antibody binding event propagates through closely arranged capturing antibodies on a sensor's gate surface. See, for example, Macchia E, Torricelli F, Caputo M, Sarcina L, Scandurra C, Bollella P, Catacchio M, Piscitelli M, Di Franco C, Scamarcio G, Torsi L. Point-Of-Care Ultra-Portable Single-Molecule Bioassays for One-Health. Adv Mater. 2024 March;36 (13): e2309705. doi: 10.1002/adma.202309705. Epub 2023 Dec. 27. PMID: 38108547.

If the sensor's gate surface (detection area) is densely packed with capturing antibodies, this dense packing allows the field of the capture molecules to electrostatically influence each other. To maximize this effect, the surface chemistry at the detection area can be engineered so that the immobilized capture molecules retain their orientation and packing density.

When a target molecule, such as an antigen or another antibody-specific ligand, binds to one of the capturing antibodies, this event induces a conformational and/or electrostatic change in that antibody. Initially localized, this change has the potential to influence nearby antibodies due to the proximity facilitated by the dense packing. The initial electrostatic change can propagate through the layer of antibodies in a domino effect; the change in one antibody affects its nearest neighbors, which in turn affect their neighbors, and so on. This propagation can be facilitated by several mechanisms: electrostatic forces between charged groups on the antibodies can transmit changes in electrostatic potential through the layer, the binding event may cause physical movements or conformational changes in the bound antibody that could mechanically influence adjacent antibodies, and antibodies have dipole moments where the alteration in the orientation or environment of one antibody's dipole upon binding can influence the dipoles of neighboring antibodies.

This propagated electrostatic change alters the overall electronic characteristics of the gate surface, specifically affecting its capacitance and conductance. These changes can then be amplified and detected as the output signal of the sensor device. The ability to detect a change initiated by a single molecular binding event could tremendously enhance the sensor's sensitivity. Instead of requiring multiple binding events across the sensor surface to generate a detectable signal, the propagation effect means that a single event can lead to a measurable change, pushing the limits of detection down to the single-molecule level. This mechanism would enable ultra-sensitive detection capabilities necessary for applications requiring the identification of very low concentrations of biomarkers, such as in early-stage disease diagnosis or in environmental monitoring where the target molecules may be present in trace amounts.

FIG. 32 stylistically illustrates a target molecule binding to one or more capture molecules, demonstrating the propagation of charge effects through the field of capture molecules on a sensor's surface. The system comprises capture molecules (3204), a die (3206), and a visualization of the propagation of the binding event (3208).

The propagation of charge effects results from a complex interplay of physical and chemical interactions that are important for the high sensitivity of biosensors in detecting target molecules. This propagation may depend on the physical arrangement of the capture molecules, their chemical nature, and the operational environment of the sensor.

When a target molecule binds to capture molecules, several fundamental interactions and phenomena occur. The binding can induce conformational changes in the capture molecule, altering its electronic properties such as charge distribution. This alteration can affect neighboring molecules through electrostatic forces. If the capture molecules are densely packed, these electrostatic changes can propagate like a wave across the capture molecule field, altering the local electrical environment and potentially influencing further molecular interactions. Additionally, biological molecules typically possess dipole moments; a change in the orientation or conformation of one molecule can trigger a chain reaction of dipole realignments, further propagating charge alterations throughout the field of capture molecules.

In sensors that utilize conductive polymers or semiconducting materials, binding events can enhance or restrict charge transfer between molecules. This influence may result from the creation or disruption of conjugation pathways, thus affecting the molecular layer's overall conductivity. Especially in FET-based sensors, the field effect induced by the binding event significantly affects the conductivity between the source and drain of a transistor. The change in surface potential at the sensor interface, caused by the binding event, modulates the transistor channel's current flow. This modulation might directly result from the propagated charge effects.

Binding events can also alter the capacitive properties of the sensor surface. Detecting changes in capacitance allows the sensor to infer the occurrence and magnitude of binding events, providing a clear indication of the propagation of charge effects through the molecular field.

FIG. 33 shows the formation of an array of drain conductors of a reconfigurable bare die semiconductor sensor. A pattern of drain electrodes 3304 is formed on the surface of a suitable semiconductor substrate.

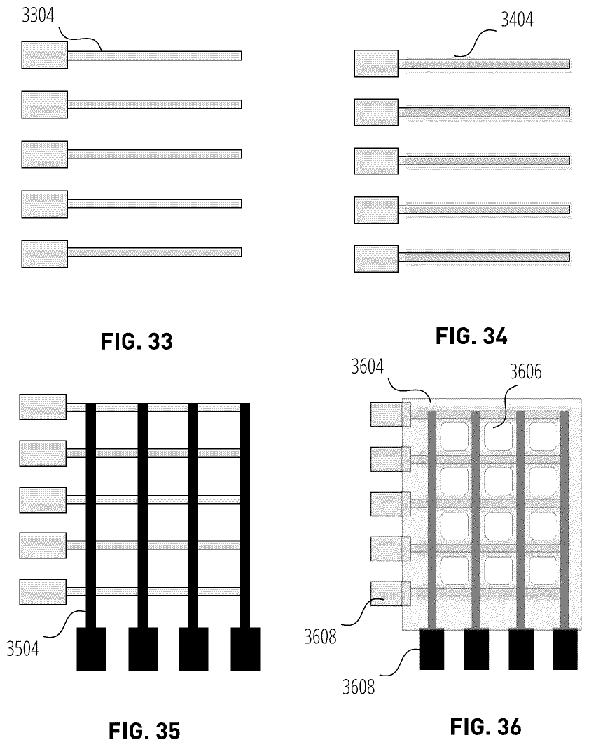

FIG. 34 shows the formation of a drain insulation layer on the an array of drain conductors of the reconfigurable bare die semiconductor sensor. A pattern of drain insulation 3404 is formed over at least of portion of the array of drain conductors. The drain insulation allows for a subsequent formation of conductors that cross over the drain conductors, where drain insulation prevents shorting between the formed patterns of conductors.

FIG. 35 shows the formation of an array of source conductors at a right angle to the array of drain conductors and over the drain insulation layer of the reconfigurable bare die semiconductor sensor. The source electrodes 3504 are formed in a pattern that intersects the drain conductors with the drain insulation preventing shorting between the formed patterns of conductors.

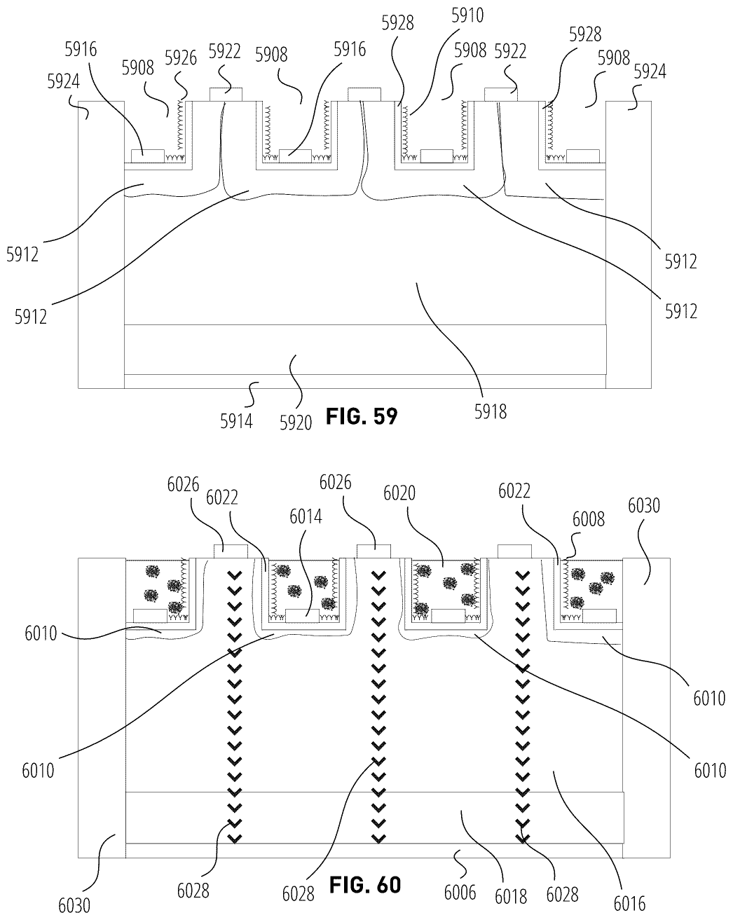

FIG. 36 shows a barrier layer formed over the arrays of drain and source conductors and having an opening for forming an array of detection areas. After forming the conductors, a layer of glass 3604 is formed so that a detection area 3606 is left exposed that defines a sample well for each sensor device. The glass layer passivates the features formed on the wafer and leaves the samples well (detection area) and bond pad 3608 for the conductors exposed. Other semiconductor sensor features, such as a gate insulator, can be formed to complete the fabrication of an array of semiconductor sensors. Also, the different functionality of the conductors (e.g., source, drain, gate) can be altered depending on the type of semiconductor sensor device architecture. For example, in the case of vertical GaN sensor structure, the intersecting conductor structure shown may form the source and gate conductors instead of the drain and source conductors as described, and the drain conductor can be provided on the backside of the GaN device substrate (see, for example, FIG. 59).

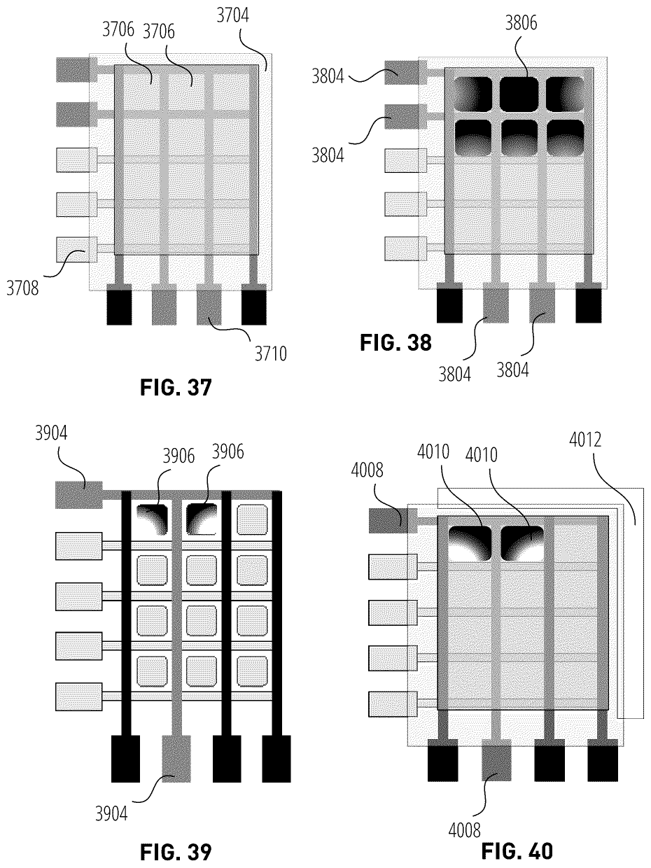

FIG. 37 shows a detection area field formed over the arrays for drain and source conductors of the reconfigurable bare die semiconductor device. The sensor array include a layer of gate insulation 3704 that is formed with over the glass layer shown in FIG. 36 or otherwise so that the detection areas 3706 have an insulator as necessary to form a field effect transistor with the individually addressable source electrodes 3710 and drain electrodes 3708 so that an x-y scanning scheme can be used to address individual or gangs of electrodes.

FIG. 38 shows a pair of drain conductors and a pair of source conductors being tapped for electrical measurement of the source to drain current flow. For example, the electrical characteristics of the detection areas measured at test points 3804 will be indicative of a measured binding events 3806 that occur at a number of detection areas as illustrated. The signal at the measured test points may be considered to have a gradient of signal strength that depends on the location of the binding event and the proximity to drain and source electrodes. This enables the possibility of obtaining a significant amount data by scanning and measuring the individually addressable electrodes over time. This data can be analyzed by AI-agents to discern patterns of useful information that indicate improved test results, improvement to the test device structure, hardware and software, better patient outcomes and lower costs.

FIG. 39 shows a construction of the array of sensor devices with a glass passivation layer defining individually addressable detection areas between the grid of source and drain electrodes. Test points 3904 are tapped to obtain an electrical signal that is indicative of binding events occurring at detection areas that have at least one edge adjacent to a tapped test point. By scan testing the drain and source electrodes of the sensor array, a complex pattern of the binding events may result which can be provided as data for AI-analysis. As an example, contrived samples of a target molecule can be used to create a training database of measurements so that unsupervised learning algorithms can analyze the complex patterns and improve the sensor's detection accuracy over time. These algorithms help to identify and classify different binding events based on the electrical signals, enhancing the system's ability to detect subtle variations in target molecule concentration and presence. This AI-driven approach allows for the adaptive recognition of specific biomolecular interactions, significantly increasing the diagnostic capabilities of the sensor array. By continuously updating the training database with new data, the system can adapt to new conditions and variations in target molecules, ensuring robust and precise diagnostics in a variety of medical and environmental applications.

FIG. 40 shows test points 4008, a measured binding event 4010, and a liquid gate electrode 4012. The liquid gate utilizes a liquid sample that covers the detection areas of the sensor array. A gate voltage applied through the liquid gate electrode to the drain electrode enables the measurement of the source-to-drain current, which varies based on both the applied gate voltage and charges influenced by binding events. These events induce a field effect that modifies the source-to-drain current flow. By analyzing changes in this current attributable to field effects, the presence or absence of a target molecule in the sample can be ascertained. The system is calibrated by comparing source-to-drain currents at specific gate voltages, using AI-analysis to discriminate and categorize different known concentrations of a target molecule based on distinct current change patterns, where all detection surfaces are functionalized with the same capture molecule. This approach enhances detection accuracy and specificity.

The method for fabricating a multi-biomarker detecting array of semiconductor sensors on a bare die involves several steps to construct and optimize the sensor's functionality. Initially, a semiconductor substrate is provided. On this substrate, a plurality of drain electrodes are formed, over which a drain insulation layer is deposited to prevent electrical shorting. Source electrodes are then formed orthogonal to the drain electrodes and positioned over the drain insulation layer.

A barrier layer is subsequently deposited over both the source and drain electrodes, designed to leave openings that define an array of detection areas. A glass layer is defined over the barrier layer, forming sample wells at each detection area while simultaneously exposing the bond pads for the source and drain electrodes, facilitating connections for signal readout.

Over this glass layer, a gate insulation layer is formed, configuring the detection areas to function as field effect transistors with individually addressable source and drain electrodes, allowing for precise control and measurement of electrical properties during sensor operation.

Additionally, a liquid gate electrode may be formed over the detection areas to facilitate the measurement of source-to-drain currents that are modified by binding events within the detection areas. This feature is crucial for sensing applications where the presence of specific biomarkers alters the electrical conductivity between the source and drain, indicative of binding events.

Furthermore, the method includes tapping pairs of source and drain conductors for electrical measurement. This allows for the analysis of binding events based on a gradient of signal strength, which is determined by the proximity to the source and drain electrodes. This configuration enables the detection of minute changes in conductivity caused by molecular interactions within the detection areas, enhancing the sensor's utility in detecting multiple biomarkers.

Note that the sequence and layers may be altered depending on the desired performance and use-case of the multi-biomarker detecting array.

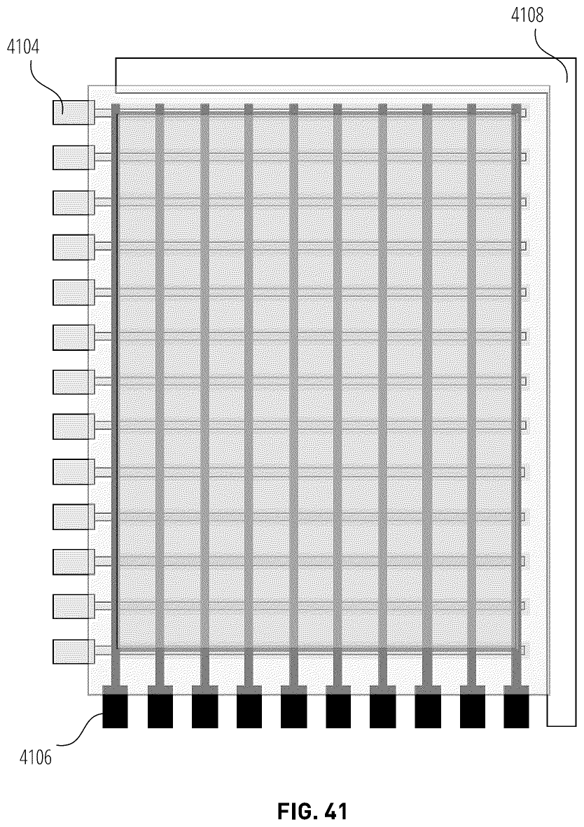

FIG. 41 shows a sensor array comprising 120 individually addressable sensor devices, organized with twelve drain electrodes 4104 and ten source electrodes 4106, with a liquid gate electrode 4108. This modular configuration, illustrated here, can be adapted in terms of the number of electrodes and their layout, allowing for different geometrical arrangements to optimize sensor functionality. The design benefits from being highly scalable, making use of standard semiconductor fabrication methods to produce large-scale arrays directly on semiconductor wafers.

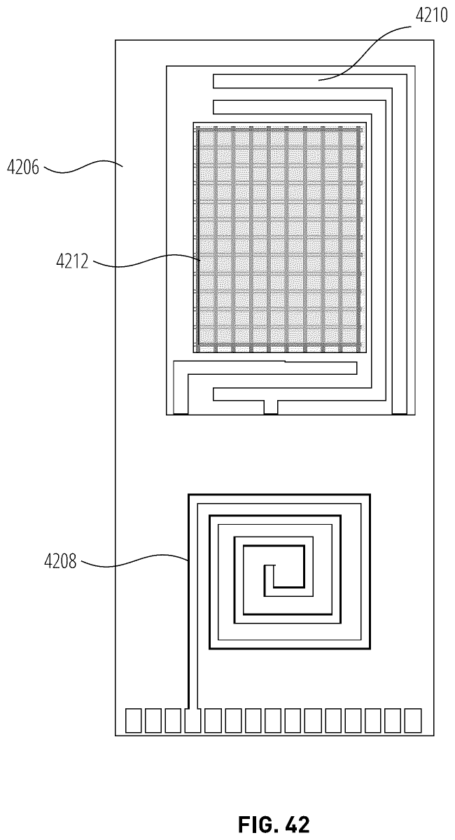

FIG. 42 illustrates a multifunctional biosensor card 4206 that integrates a variety of advanced features for enhanced diagnostic capabilities. The features includes an antenna 4208 for wireless communication of test results and for energy harvesting from an RF signal. The biosensor card also incorporates liquid detection features 4210 for detecting the flow of a fluid sample before and after flowing over the biosensor device array.

The bare die biosensor array 4212 consisting of 120 individually addressable biosensor devices. These devices can be specifically tailored for diverse diagnostic purposes: each sensor can be functionalized with one or more different capture molecules, allowing the card to perform comprehensive panel tests for a variety of target analytes simultaneously. This capability makes it particularly valuable in settings where multiplex testing is required, such as detecting multiple pathogens or biomarkers from a single sample.

Alternatively, the sensors can be uniformly functionalized with the same capture molecule across the entire array, transforming the device into a wide-area field-effect transistor (FET) sensor. This configuration maximizes the surface area available for binding events, significantly enhancing the probability of detecting low-concentration target molecules in the sample. Such a setup is ideal for applications requiring high sensitivity and specificity, like early disease detection or environmental monitoring.

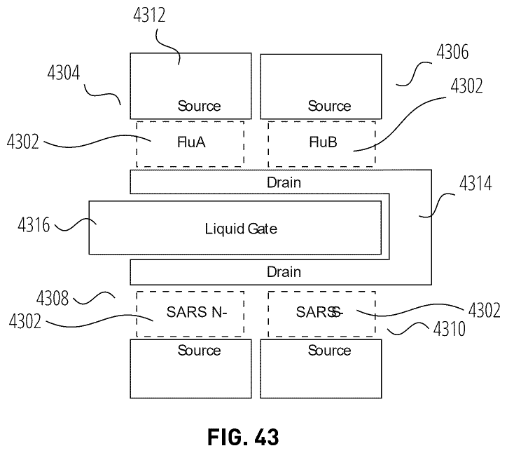

FIG. 43 illustrates a bare die biosensor having individually addressable sensor devices functionalized for FluA/FluB/SARS S/SARS N virus testing. In this case, each sensor device on the bare die semiconductor device is functionalized for detecting if the test sample contains a biomarker of FluA, FluB or SARS. In this example, the biosensor consists of a sensor device semiconductor device, however other semiconductor structures, such as a GaN or gFET biosensor could be utilized, or printed electronic, electro-chemical, LFAs, or other biosensors could be utilized by a diagnostic test system.

In this example, the SARS biomarkers include both the SARS N-protein and S-protein. Each charge transfer layer of the different gFETs has a different type of capture molecule (e.g., capture molecule or detecting FluA biomarker). The capture molecules are immobilized at the detection area of each corresponding gFET. A liquid gate electrode, a drain electrode, and a source electrode provide electrical conduction to the semiconductor features that form the different biosensor ganged on semiconductor bare die, where one or more of these biosensor can be functionalized at the processed wafer level.

The present invention is designed as a modular system of subassemblies, each module can be separately completed and tested to ensure their functionality before being integrated into the diagnostic system. This modular approach facilitates rapid reconfiguration of the diagnostic system to respond to new pathogen threats, new disease use-cases and rapid implementation of improvements developed by engineers with AI-assisted improvement guidance. The packaged biosensor can have multiple biosensors on a single bare die, and each sensor can be individually functionalized with a different capture molecule. This allows for the creation of a syndromic biosensor that can test for multiple biomarkers of the same disease (e.g., S and N proteins and even RNA of SARS-COV-2 virus) and/or different diseases (e.g., SARS, Flu-A, Flu-B).

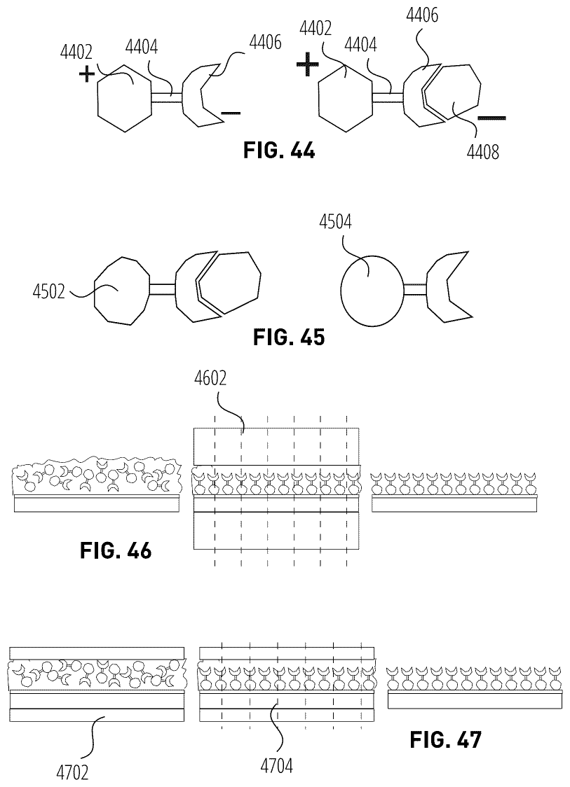

FIG. 44 illustrates an applied-field-reactive capture molecule conjugate having an applied-field-responsive end and a capture molecule end with a linker molecule providing electro-chemical properties that change at least one of a polarity and a conductivity. The system comprises a field reactive particle 4402, a linker 4404, a capture molecule 4406, and a target molecule 4408.

FIG. 45 illustrates a capture molecule conjugate having a magnetically reactive end and a capture molecule conjugate having an electric field reactive end. The system comprises a magnetic reactive particle 4502 and a field reactive particle 4504.

FIG. 46 shows a process for forming aligned and oriented capture molecule conjugates aligned in a magnetic field on a dissolvable adhesive or lateral flow assay membrane or other support structure. The system comprises an aligning field 4602.

FIG. 47 shows a process for forming aligned and oriented capt molecule conjugates aligned in an elec field on a dissolvable adhesive or lateral flow assay membrane or other support structure.

The system comprises a magnetic field plate 4702 and an aligning field 4704. In accordance with an exemplary embodiment, an array of applied-field-reactive capture molecule conjugates is made by providing a dissolvable adhesive film or other support film or structure, on a substrate, membrane, liner, or free standing. A carrier fluid that is a non-solvent for the dissolvable adhesive film has randomly dispersed applied-field-reactive capture molecule conjugates. An aligning field is applied to the carrier fluid for assembling the applied-field-reactive capture molecule conjugates onto the dissolvable adhesive film. The carrier fluid is removed (evaporated, dip or spin coating) leaving the assembled applied-field-reactive capture molecule conjugates fixed on the support film.

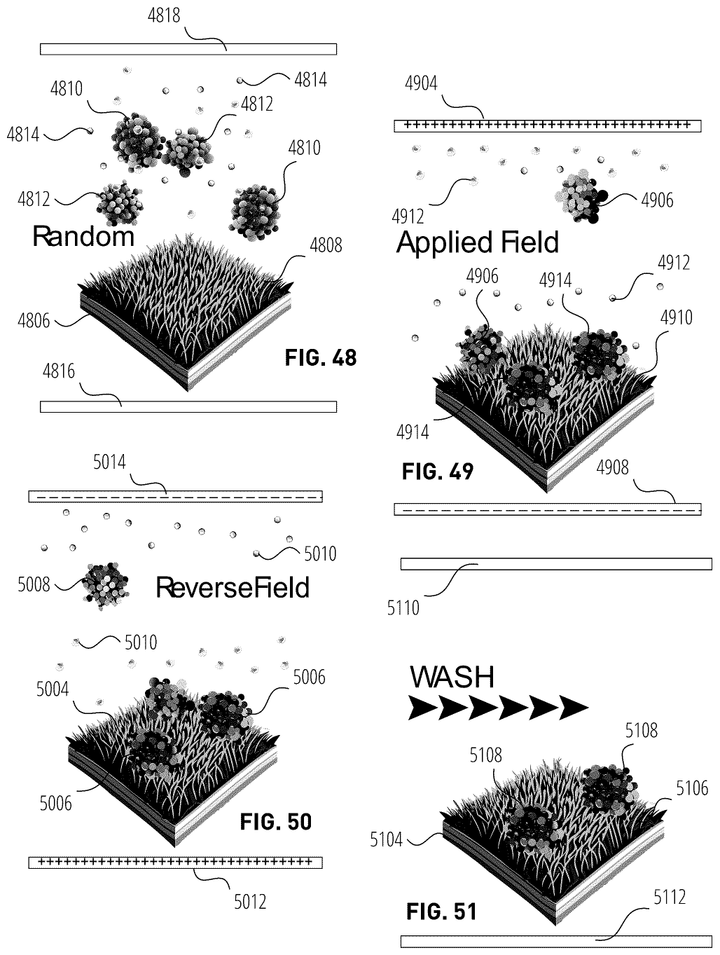

FIG. 48 illustrates a bare die sensor having a detection area covered by a field of capture molecules, and a liquid sample containing specific target molecules and non-specific ions and molecules. The system comprises a bare die 4806, a capture molecules 4808, a target molecule 4810, a non-target molecule 4812, an ion 4814, a bottom driving electrode 4816, and a top driving electrode 4818.

FIG. 49 illustrates electric field applying top and bottom driving electrodes applying an electric field causing the specific target molecules and non-specific ions and molecules in the liquid sample to orient and migrate in a direction depending on the positive and negative charges of the molecules and ions, where the capture molecules and target molecules are brought into the potential for a specific binding event. The system comprises a top driving electrode 4904, a non-target molecule 4906, a bottom driving electrode 4908, a capture molecules 4910, an ion 4912, and a target molecule 4914.

FIG. 50 shows the applied electric field being reversed causing the molecules and ions to orient and migrate in another direction where the target molecules that bind with the capture molecules remain immobilized at the detection area. The system comprises a capture molecules 5004, a target molecule 5006, a non-target molecule 5008, an ion 5010, a bottom driving electrode 5012, and a top driving electrode 5014.

FIG. 51 shows an optional step of washing away non-specific ions and molecules that are not bound to capture molecules to improve the signal caused by target molecules that remain immobilized at the detection area. The system comprises a bare die 5104, a capture molecules 5106, a target molecule 5108, a top driving electrode 5110, and a bottom driving electrode 5112.

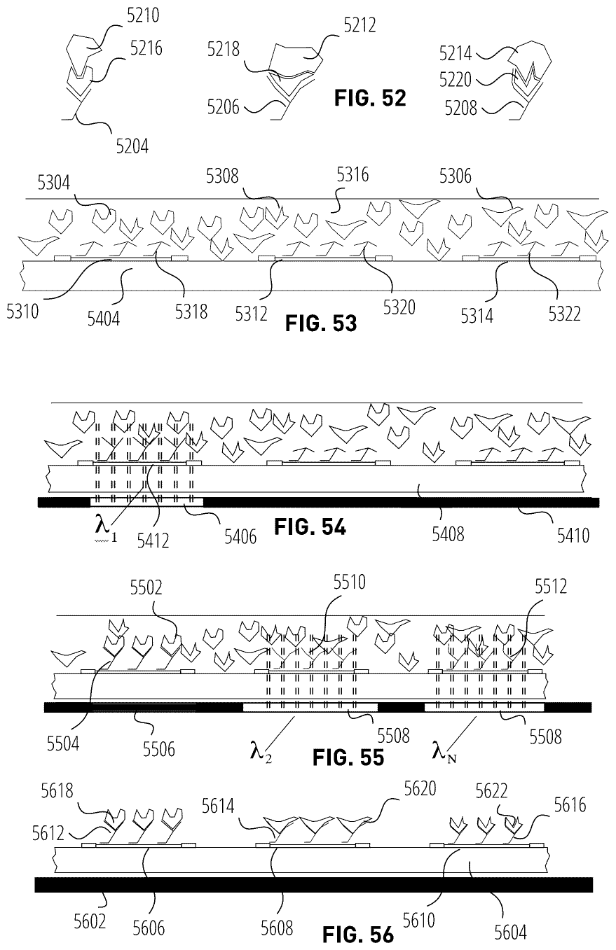

FIG. 52 illustrates different capture molecules for detecting different capture molecules and bond to activatable linkers. In this embodiment, an unactivated linker is selectively activate by patterned radiation to bind different capture molecules to functionalize different sensor devices formed on a semiconductor wafer. The capture molecules include two or more different types (e.g., capture molecule1, capture molecule2, and capture molecule3. Each capture molecule selectively binds with a different target molecule (respectively, target molecule1, target molecule2, target molecule3). The detection interface of different sensor devices on the same wafer can be functionalized with a distinct capture molecule that can be used to detect the presence of a class of biomarkers. For example, in the case of SARS corona viruses, the N-protein within the viral envelope of these virus is usually preserved from variant to variant, while the S-protein will mutate and become a variant or sub-variant of a predecessor variant or original viral strain. The N-protein biomarker can then be considered a class of biomarker that is detectable for many different strains of SARS.

The same detection interface of a semiconductor sensor, or more detection interfaces of adjacent sensors, can be functionalized with multiple capture molecules. For example, if the same detection interface has multiple capture molecules immobilized on the same charge transfer layer, a screening test can be used where a detected change in signal output caused by capturing at least one type of biomarker indicates a potential health condition.

As a specific but non-limiting example, researchers have identified these six different biomarkers that indicate a potential risk of pancreatic cancer: ApoA1, CA125, CA19-9, CEA, ApoA2, and TTR (see, Kim H, Kang K N, Shin Y S, et al. Biomarker Panel for the Diagnosis of Pancreatic Ductal Adenocarcinoma. Cancers (Basel). 2020; 12 (6): 1443. Published 2020 Jun. 1. doi: 10.3390/cancers12061443). One or more electronic biosensor interface can be functionalized with two or more types of capture molecules that selectively bind to a respective one of these six identified biomarkers. Gastro-intestinal lavage obtained during a colonoscopy can be used as a biosample that is tested. A screening test for pancreatic testing can use a change in the signal output from the biosensor that exceeds a predetermined threshold as an indication that the test subject may have pancreatic cancer and should be tested further. Similarly, the detection interface of six adjacent semiconductor sensors can be functionalized with a respective capture molecule that selectively binds with one corresponding biomarker. Probabilistic analysis of the signal out from each sensor can be used to determine if the test subject should undergo additional testing for pancreatic cancer.

FIG. 53 shows selectively binding a first sub-set of capture molecules to activatable linker molecules. The system comprises a capture molecule1 5304, a capture molecule2 5306, a capture molecule3 5308, a sensor1 5310, a sensor2 5312, a sensor3 5314, a liquid medium 5316, a linker1 5318, a linker2 5320, and a linker3 5322.

A plurality of sensor devices are formed on a semiconductor substrate wafer, each sensor device includes a source, a drain and at least one channel region. At least one of an insulator layer and a dielectric layer is formed over each channel region and a detection area including a charge transfer layer is formed adjacent or near to at least one of an insulator layer and dielectric layer. That is, the sensor devices can have a range of device configurations and material layers, including sensor constructions known as g-FETs (graphene field effect transistors), GaN HEMT, and vertical GaN, etc., as a few examples of known semiconductor device constructions.

For the semiconductor construction of an optimized sensor, ag-FET sensor performance parameters include, for example: the dain current, ID, the trans-conductance, gm, the channel conductance, gD, the threshold voltage, VT, the gate stack reliability, and the gate direct tunneling current density, JDT. Most of these parameters are influenced by the gate dielectric capacitance, Cdi, channel mobility, Ich, metal-semiconductor work function difference,/MS, gate stack charge density, Qgsc, interface trap density, Dit, and bulk dielectric trap density, Dbt, cf. (see, for example, High Permittivity Gate Dielectric Materials, Samares Kar, DOI 10.1007/978-3-642-36535-5)

The gate stack capacitance influences many important aspects of the g-FET sensor. To improve the permittivity at the charge transfer layer, and the sensor performance characteristics such as sensitivity, the insulator/dielectric layer can be a multi-layered stack with a high-k material core, including at least one of HfO2, La2O3, HfSiO, HfAIO, HfNO, HfSiON, ErTiO5, SrTiO3, LaScO3, LaA103, GdScO3, LaLuO3, La2Hf2O7, Gd2O3, La2SiO5, SrHfO3.

An unactivated linker is incubated and immobilized on the charge transfer layers of the g-FETs. Capture molecules are selectively immobilized on the charge transfer layers of the g-FETs. Different types of capture molecules can be immobilized on the different g-FETs using selective photo-activation of the unactivated linker molecules. The activatable linker molecules are first immobilized on the charge transfer layers (or detection area) through an incubation step as described herein. A capture molecule carrier fluid containing the capture molecules as free-floating capture molecules is disposed over a top surface of the semiconductor substrate wafer covering the plurality of device regions. For example, spin and/or dip coating can be used to form a thin film of the carrier fluid as a liquid medium containing a first type of free-floating capture molecules (capture molecule1) on the surface of the wafer (step one). Prior to activation the activatable linker molecules are relatively less receptive to binding to the free-floating capture molecules. In practice, it is important that the chemistry that causes the capture molecule to bind to the linker molecule can be initiated by a selective process, such as patterned photo-radiation. For example, the photo-radiation can be irradiated using an emission plate through a transparent substrate and used to selectively activate the activatable linker molecules to form activated linkers immobilized at some of the charge transfer layers or detection areas (step two). The activated linkers are receptive to binding with the free-floating capture molecules while the unactivated linkers do not bind with the capture molecules. Once the pattern of activatble linker molecules is formed, incubation at an appropriate time and temperature is used to bind the capture molecules selectively to the activatble linker molecules (step three).

FIG. 54 illustrates the steps of selectively binding a first sub-set of capture molecules to activatable linker molecules. The system comprises a transparent substrate 5404, an emitting pixel 5406, a transparent substrate 5408, an emission plate 5410, and a sensor1 5412.

FIG. 55 illustrates illustrates the steps of selectively binding a respective second and third sub-set of capture molecules to corresponding activatable linker molecules. The system comprises a capture molecule1 5502, a linker1 5504, an emission plate 5506, an emitting pixel 5508, a linker2 5510, and a linker3 5512.

FIG. 56 shows three sensors formed on a wafer having a transparent substrate, each respective sensor having a corresponding selectively activate-able capture molecule and linker pair immobilized on its detection area. The system comprises an emission plate 5602, a transparent substrate 5604, a sensor1 5606, a sensor2 5608, a sensor3 5610, a linker1 5612, a linker2 5614, a linker3 5616, a capture molecule1 5618, a capture molecule2 5620, and a capture molecule3 5622.

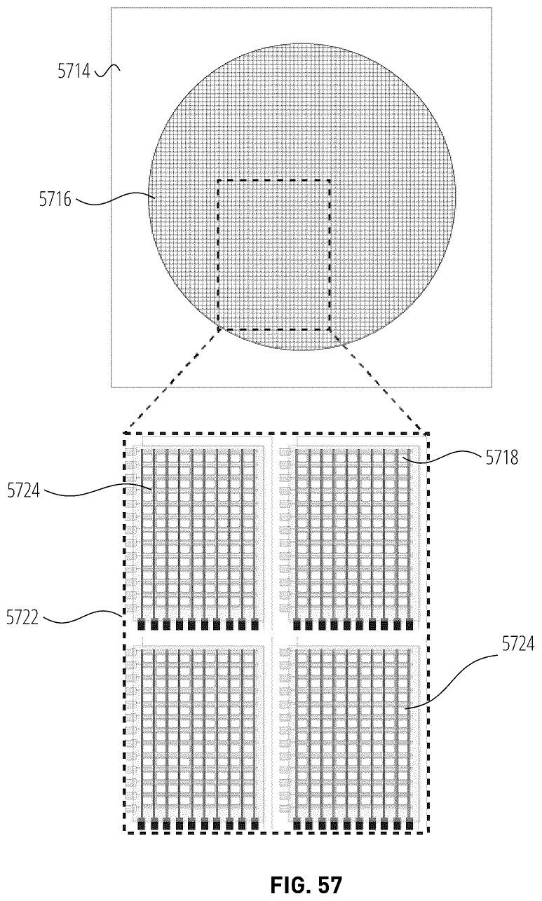

FIG. 57 illustrates an emission plate with individually addressable emitting pixels for selectively immobilizing capture molecules on respective detection areas of a biosensor array. The system comprises an emission plate 5714, an emitting pixel 5716, a detection area 5718, a section of wafer 5722, and a biosensor array 5724.

The described method involves fabricating a multi-biomarker detecting semiconductor sensor array on a wafer with a transparent substrate, such as quartz or sapphire. The process includes forming an array of sensor devices, each comprising a source, a drain, and at least one channel region, over which a gate oxide layer is formed. An array of detection areas is established on top of the gate oxide layer.

The unique aspect of this method is the immobilization of capture molecules on these detection areas through a multi-step process. Initially, a first set of activatable linker molecules is immobilized on the detection areas. These linkers are designed to be activated by a specific wavelength of radiation. A carrier fluid containing the first set of free-floating activatable capture molecules is then applied over the wafer. Selective irradiation through the transparent substrate activates the linkers, binding them to the first set of capture molecules. The carrier fluid is subsequently removed, and the surface is rinsed to leave behind the immobilized capture molecules. This process can be repeated for additional sets of capture molecules, each activated by a different wavelength, to enable the detection of various target molecules simultaneously. The activatable linkers can be provided all at once, or provided during the repeated process steps for the additional sets of capture molecules.

Further steps may include washing away unbound molecules after each step for selectively binding the capture molecules. Each device region can independently detect different target molecules, allowing for simultaneous multiplex testing. The sensors are calibrated based on known concentrations of target molecules to establish detection thresholds, ensuring accurate and reliable diagnostics.

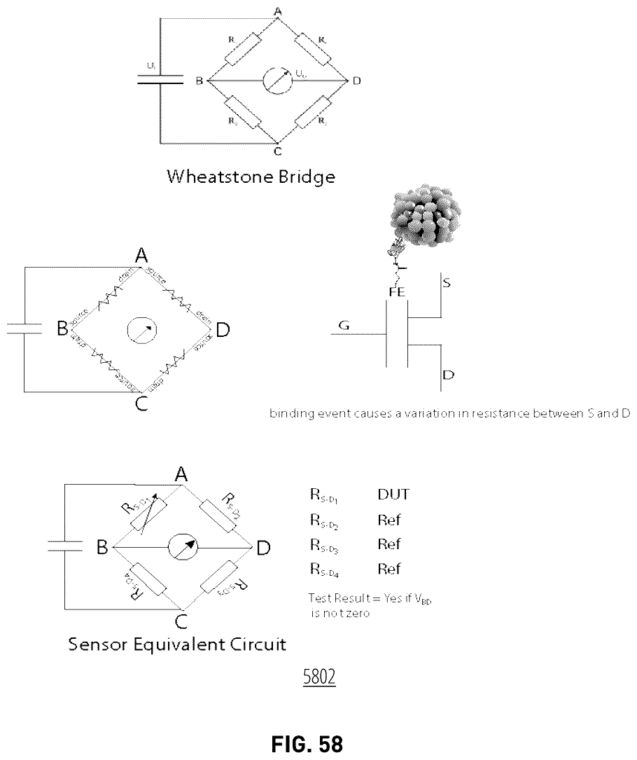

FIG. 58 shows a Wheatstone Bridge circuit concept utilizing semiconductor sensors, where each sensor's source-to-drain resistance acts as a bridge resistor, enabling the detection of molecular binding events that cause measurable changes in resistance in the S-D resistance of a DUT (device under test). The system comprises an arrangement of individual semiconductor sensors to form a Wheatstone Bridge. A Wheatstone Bridge is a fundamental electrical circuit used to measure very small changes in resistance. It operates on the principle of balancing two legs of a bridge circuit, one leg of which includes the component to be measured. A typical Wheatstone Bridge consists of four resistors arranged in a diamond shape. These four resistors include three resistors of known value and fourth resistor whose value is to be determined or monitored. The bridge has two input terminals connected to a power source and two output terminals that connect to a measuring device. The bridge is balanced when the voltage across the two output terminals is zero. This is achieved when the ratio of the resistances in one pair of opposite arms equals the ratio in the other pair. When this condition is met, there is no voltage difference between the midpoints of these two resistor pairs, and the bridge is said to be “in balance”. When the resistance of one or more changes and disturbs the balance of the bridge is disturbed, a detectable voltage difference is developed across the output terminals of the bridge. The voltage difference across the output terminals is proportional to the difference between the ratios of the two resistor pairs. The sensitivity of a Wheatstone Bridge makes it suitable for detecting very small changes in resistance. The sensitivity can be enhanced by carefully selecting the values of the resistors to maximize the output voltage for a given change in variable resistance that is being detected.

FIG. 58 illustrates an application of the Wheatstone Bridge concept using semiconductor sensor devices, integrating the principles of this classical electrical circuit to measure subtle changes in resistance for environmental and biosensing applications. The depicted Wheatstone Bridge circuit consists of four resistors configured in a diamond layout, typically comprising three known resistors and a fourth variable resistor (DUT-Device Under Test), which represents the resistance between the source(S) and drain (D) of a semiconductor sensor affected by a binding event.

In operation, the bridge is initially balanced with no voltage difference between the middle points of the resistor pairs, indicating equilibrium when the resistance ratios across the bridge are equal. Upon exposure to a sample, if a target molecule binds to the sensor area, this event alters the S-D resistance of the DUT, subsequently disturbing this balance. This imbalance introduces a measurable voltage difference across the bridge's output terminals, which is directly proportional to the magnitude of resistance change due to the binding event. The enhanced sensitivity of this Wheatstone Bridge setup, as shown in FIG. 58, allows for the detection of very small changes in the S-D resistance due to molecular interactions at the sensor's detection surface.

In accordance with a non-limiting exemplary embodiment, a method employs a Wheatstone Bridge circuit for the detection of molecular binding events using semiconductor sensors, each equipped with a source and a drain. This setup arranges the sensors in such a way that each sensor's source-to-drain resistance acts as a resistor within the bridge. The Wheatstone Bridge is then connected to a power source and a voltage measurement device across two output terminals. Initially, the bridge is balanced to ensure that no voltage difference exists across these terminals under baseline conditions, where no target molecules are bound to the sensors.

Once the semiconductor sensors are exposed to a sample that potentially contains target molecules, any molecular binding events alter the source-to-drain resistance of one or more sensors. This change disrupts the balance of the bridge, resulting in a measurable voltage difference across the output terminals. This voltage difference is indicative of molecular binding events. To enhance detection sensitivity, the resistance values of the known resistors within the bridge are adjusted, and the bridge can be calibrated under various baseline conditions to account for environmental or sensor variations. The molecular binding typically involves interactions between capture molecules, which are immobilized on the sensors, and target molecules within the sample. The configuration includes three resistors with known resistances and a variable resistor that dynamically changes due to molecular binding events, thus allowing precise measurements of these interactions.

FIG. 59 illustrates a semiconductor sensor device configuration that utilize a vertical GaN semiconductor architecture.

A vertical GaN (Gallium Nitride) semiconductor architecture refers to a design where the electron flow (current) is perpendicular to the surface of the wafer. Unlike lateral structures where devices are fabricated side by side on the surface of the semiconductor substrate, vertical architectures stack the components such as source, drain, and gate vertically. This configuration allows for devices that can handle higher power and voltage levels due to the ability to spread heat more efficiently and use the bulk of the material for current conduction. Vertical GaN structures are particularly advantageous in power electronics, enabling compact, efficient, and high-performance devices suitable for applications like power conversion systems, electric vehicles, and renewable energy technologies.

The sensor system comprises capillary channels 5908, capture molecules 5910, depletion layers 5912, a drain 5914, gates 5916, an N−GaN drift layer 5918, a N+GaN wafer 5920, sources 5922, termination edges 5924, capture molecules 5926, and pGaN layer 5928. The depletion layers extend through the semiconductor bulk and pinch off current from flowing vertically between the sources and the drain.

The vertical GaN (Gallium Nitride) semiconductor sensor array is designed for advanced sensing applications. Depletion Layers 5912 that extend from pGaN 5928 form around the sensor elements where charge carriers are absent for controlling the flow of electricity through the device. The width and properties of these layers are altered by the presence of target molecules, affecting the overall electrical characteristics of the sensor. The drain 5914 is located at the base of the sensor structure and collects electrons that flow down through the device. An N−GaN drift layer 5918 is the region where charge carriers mainly move and is made of n-type GaN to create a pathway with a controlled level of electron density that can be optimized to detect a change in the field effect caused by binding events occurring between capture molecules and target molecules at the detection array of the sensor devices. An N+GaN wafer 5920 serves as the substrate for the sensor, providing a highly conductive layer that supports the overall structure and enhances the device's electrical properties. Capillary channels 5908 are formed within the sensor, these channels allow the flow of a liquid sample so that a large detection area with immobilized capture molecules can bind with target molecules contained in the fluid sample. The capture molecules 5910 are immobilized on the detection areas of the sensor devices and are designed to bind specifically with the target analytes. The binding events between these molecules and the targets initiate changes in the sensor's electrical properties. Gates 5916 can be provide to control the flow of carriers in the N−GaN drift layer. By applying different voltages to these gates, the electric field across the sensor can be precisely modulated, altering the conductivity of the N−GaN drift layer in have a detectable response to the binding events. Sources 5922 are the entry points for electrons into the sensor device and inject carriers into the N−GaN drift layer. Termination edges 5924 define the physical boundaries of the sensor elements, helping to isolate the electrical activity within each sensor and prevent cross-talk between adjacent devices. The pGaN layers 5928 are a p-type GaN layer that works in conjunction with the N−GaN drift layer to form a p-n junction. These p-n junctions provide a diode action within the sensor devices.

FIG. 60 shows the change in the depletion layer resulting from binding events between the target molecules and the capture molecules. The system comprises a drain 6006, a capture molecules 6008, a depletion layer 6010, a gate 6014, an N−GaN drift layer 6016, a N+GaN wafer 6018, a capillary channels 6020, a pGaN 6022, a source 6026, a charge conduction 6028, and a termination edge 6030. The binding events cause a field effect change in the depletion layer and opens the charge conduction path allowing more electrons to flow vertically between the sources and the drain.

The operation of the vertical GaN sensor structure depicted in FIGS. 59 and 60 involves detecting the presence of target molecules through their binding to capture molecules embedded within the sensor architecture. This detection mechanism relies on changes in the electrical properties of the sensor, particularly within the depletion layer, as a result of molecular binding events. As a starting condiction, there is no liquid sample or target molecules present (FIG. 59). The sensor is in a baseline state without any target molecules bound to the capture molecules. The depletion layers around the pGaN and N−GaN junction are fully formed and prevent charge carriers from moving freely across the sensor, effectively increasing the sensor's Source to Drain resistance. A voltage applied to the gates controls the width of the depletion layers, setting the baseline conductance across the sensor. The gate voltage can be set so that no significant current flows through the sensor as the conductance is mainly blocked by the depletion layers.

In FIG. 60, target molecules present in the environment interact and bind with the capture molecules 6008 located within the capillary channels 6020. These channels direct the liquid sample with the target molecules to the active sensor areas. The binding of target molecules to the capture molecules introduces a local field effect. This effect alters the electric field at the interface of the pGaN 6022 and the depletion layers 6010. The local field effect caused by the binding modulates the width of the depletion layers 6010 and allows more charge carriers to flow through the sensor, particularly in the vertical direction from the source 6026 to the drain 6006. The reduction in the depletion layer width opens up a conduction path 6028 for charge carriers with an increase in current flow between the source 6026 and the drain 6006, which can be measured as a decrease in the overall resistance of the sensor (e.g., in conjunction with the Wheatstone Bridge arrangement shown in FIG. 58.

The increase in current flow directly correlates to the presence of target molecules. The amount of current change provides quantitative information about the concentration of target molecules bound to the capture molecules. The changes in current are processed and translated into a measurable signal, indicating the presence and possibly the concentration of target molecules. The vertical GaN sensor structure utilizes the change in electrical properties caused by molecular binding events to detect the presence of specific target molecules. This process leverages the vertical architecture of the sensor to effectively modulate charge carrier flow and sensitivity, enhancing the sensor's performance in detecting molecular interactions.

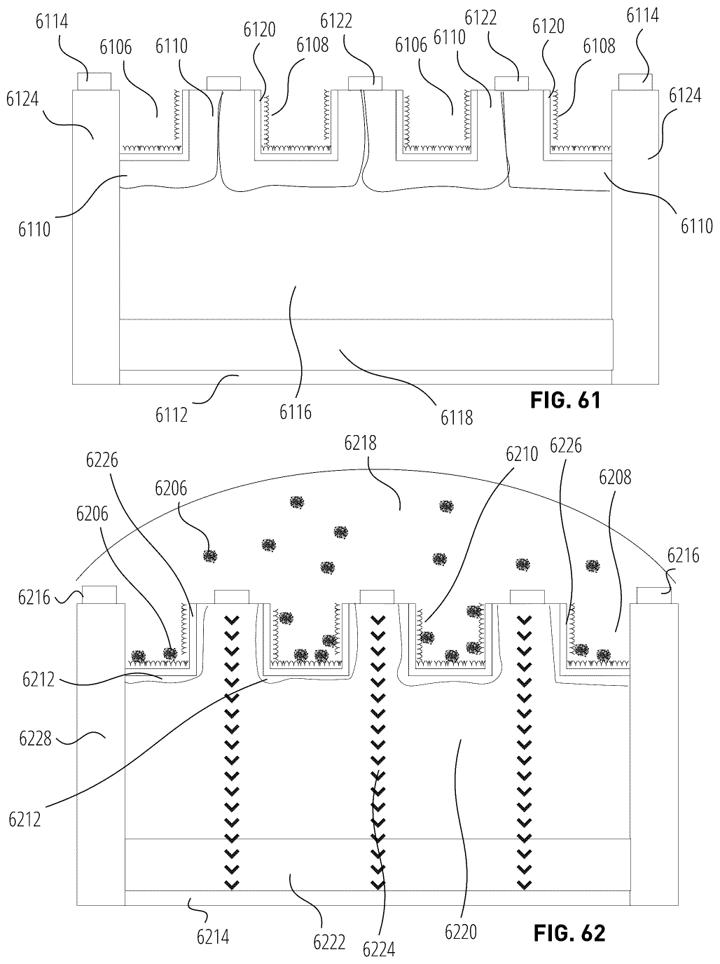

FIG. 61 is similar to FIG. 59 but includes one or more liquid gate electrodes that allow a gate voltage to be applied through a liquid sample disposed at the detection area of the sensor device. The system comprises a capillary channels 6106, a capture molecule 6108, a depletion layer 6110, a drain 6112, a liquid gate electrode 6114, an N−GaN drift layer 6116, a N+GaN wafer 6118, a pGaN 6120, a source 6122, and a termination edge 6124.

FIG. 61 presents a vertical Gallium Nitride (GaN) semiconductor sensor structure designed for enhanced detection of chemical or biological targets through an innovative liquid gate electrode mechanism. This design is an evolution of the typical vertical GaN sensor, incorporating liquid gate electrodes to modulate device properties directly via a liquid medium. Below is a detailed breakdown of each component and its function within the system. Capillary channels 6106 are provided to facilitate the movement of liquid samples towards the sensor's active area. They are critical for ensuring that the liquid sample, containing potential target molecules, reliably reaches the capture sites. Capture molecules 6108 are immobilized on the detection area of the sensor devices, which correspond to the walls of the capillary channels. These molecules are engineered to specifically bind to target molecules present in the liquid sample. Binding events at these sites are the chemical processes that result in detectable changes in the sensor's electrical properties.

A depletion Layer 6110 region forms around the junctions within the sensor where charge carriers are depleted when no external influence is applied. The binding of target molecules to the capture molecules affects this layer through a field effect that changes the sensor's source to drain conductivity.

In the vertical GaN structure, the drain 6112 is positioned at the bottom of the device, the drain collects carriers that flow through the device and a drain bond pad, along with the bond pads of the sources, are the test points for the measurement of changes in electrical current as a result of binding events.

A novel feature in this sensor design is the use of a liquid gate electrode 6114 along with the vertical GaN structure. The liquid gate electrode allows for the application of a gate voltage directly through the liquid sample. This innovative approach enables real-time modulation of the electrical characteristics of the depletion layer based on the chemical composition of the sample and can be used for applying an electrical field that drives target molecules towards the capture molecules and helps clear ions and non-specific molecules (non-target molecules) from the detection area.

The vertical GaN structure includes an N−GaN drift layer 6116, which is typically a lightly doped layer that supports the vertical movement of charge carriers from the source to the drain. An N+GaN Wafer 6118 provides a highly conductive pathway for charge carriers, supporting the overall architecture of the sensor and enhancing its electrical response. pGaN layer 6120 is formed as features of the sensor structure that works along with the N−GaN drift layer to create a p-n junction with a conduction channel that varies depending on the field effect caused by the binding events between the capture and the target molecules. Sources 6122 inject carriers into the N−GaN drift layer and in this configuration are located at the top of the sensor structure. The termination edges 6124 define the physical boundary of the sensor element.

In operation, the liquid gate electrode in FIG. 61 introduces a dynamic method to control the sensor's response by applying a voltage across the liquid sample itself. This setup allows for immediate adjustments in the sensor's electrical characteristics in response to the real-time chemical environment, enhancing sensitivity and selectivity. When target molecules in the sample bind to the capture molecules, they induce changes in the electric field across the depletion layer, detected as variations in current flow between the source and drain.

FIG. 62 illustrates the vertical GaN with liquid gate sensor when a liquid sample that contains target molecules is applied to the detection areas of the sensors. FIG. 62 shows how target molecules in a liquid sample influence the sensor's detection areas by binding with capture molecules, thus modulating electrical properties for enhanced signal detection. The system comprises a target molecule 6206, capillary channels 6208, capture molecules 6210, a depletion layer 6212, a drain 6214, a liquid gate electrode 6216, an EBC 6218, an N−GaN drift layer 6220, a N+GaN wafer 6222, charge conduction 6224, pGaN 6226, and a termination edge 6228. The vertical GaN sensor with the liquid gate electrode operates to detect the presence of the target molecules in the liquid sample by utilizing the interaction between the capture molecules and the target molecules to alter the electric field across the depletion layer. This alteration modifies the conductivity between the source and the drain, allowing the detection of the binding events through changes in the current flow, which is enhanced by the presence of the liquid gate electrode that applies a controlled voltage to precisely modulate the sensor's response to the detected molecules.

The described vertical GaN semiconductor sensor utilizes a layered structure and specific semiconductor design elements to enable precise molecular detection through electric field modulation in response to molecular binding events. The sensor is constructed on a N+GaN wafer that serves as a substrate, topped by an N−GaN drift layer which hosts a plurality of capillary channels. These channels guide a liquid sample directly to detection areas, ensuring targeted interaction with capture molecules immobilized within these channels. A pGaN layer, situated over the N−GaN drift layer, forms p-n junctions which play a crucial role in controlling charge carrier flow. This is achieved through the creation of depletion layers at the interfaces between the pGaN and N−GaN layers. These depletion layers are engineered to adjust their width in response to changes in the electric field caused by molecular binding, thereby modulating the conductivity within the N−GaN drift layer.

Sources positioned at the top surface of the pGaN layer are responsible for injecting charge carriers, while a drain located at the bottom portion of the N+GaN wafer collects these carriers, allowing for the measurement of flow alterations indicative of the presence and concentration of target molecules. Additionally, the sensor incorporates a liquid gate electrode which can apply and dynamically adjust a gate voltage through the liquid sample, adapting in real-time to changes in the sample's chemical composition. This feature is crucial for maintaining the precision and adaptability of the sensor to various molecular environments.

Further enhancing the sensor's functionality, termination edges around the sensor elements isolate electrical activity within each device, preventing cross-talk between adjacent sensors. This isolation is essential for maintaining the accuracy and reliability of individual sensor readings in a densely packed array. The strategic configuration of these components allows for a vertical charge carrier pathway that is optimized for high sensitivity in detecting molecular interactions. The binding events between capture molecules and target molecules induce changes in the electric field at the pGaN and N−GaN interface, which are detected as variations in current flow between the source and the drain, providing a quantifiable measure of molecular presence.

The foregoing description has provided by way of exemplary and non-limiting examples a full and informative description of the invention. However, various modifications and adaptations may become apparent to those skilled in the relevant arts in view of the foregoing description, when read in conjunction with the accompanying drawings and the appended claims. However, all such and similar modifications of the teachings of this invention will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

The foregoing description has provided by way of exemplary and non-limiting examples a full and informative description of the invention. However, various modifications and adaptations may become apparent to those skilled in the relevant arts in view of the foregoing description, when read in conjunction with the accompanying drawings and the appended claims. However, all such and similar modifications of the teachings of this invention will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1-54. (canceled)

55. A vertical GaN semiconductor sensor comprising:
a N+GaN wafer forming a substrate;
a N−GaN drift layer disposed over said N+GaN wafer;
a plurality of capillary channels defined within said N−GaN drift layer;
a pGaN layer disposed over said N−GaN drift layer, forming p-n junctions therewith;
a drain located at a bottom portion of said N+GaN wafer;
multiple sources disposed at a top surface of said pGaN layer;
a plurality of depletion layers formed at interfaces between said pGaN layer and said N−GaN drift layer, configured to control charge carrier flow based on binding events occurring at detection areas;
capture molecules immobilized within said capillary channels and configured to bind target molecules;
a liquid gate electrode configured to apply a gate voltage through a liquid sample disposed at said detection areas;
wherein said sensor is configured to detect target molecules by modulation of the depletion layers and charge carrier flow in response to binding events.

56. The vertical GaN semiconductor sensor of claim 55, wherein said capillary channels are configured to guide a liquid sample to said detection areas for interaction with said capture molecules.

57. The vertical GaN semiconductor sensor of claim 55, wherein said capture molecules are specifically configured to bind to one or more predetermined types of target molecules present in said liquid sample.

58. The vertical GaN semiconductor sensor of claim 55, further comprising termination edges around said sensor elements to isolate electrical activity within each sensor and prevent electrical cross-talk between adjacent sensors.

59. The vertical GaN semiconductor sensor of claim 55, wherein said depletion layers are configured to adjust their width in response to an electric field change caused by said binding events, thereby modulating conductivity of said N−GaN drift layer.

60. The vertical GaN semiconductor sensor of claim 55, wherein said liquid gate electrode is configured to dynamically adjust said gate voltage in response to real-time changes in said liquid sample's chemical composition.

61. The vertical GaN semiconductor sensor of claim 55, wherein said liquid gate electrode is configured to dynamically adjust said gate voltage in response to real-time changes in said liquid sample's chemical composition.

62. The vertical GaN semiconductor sensor of claim 55, wherein said sources are configured to inject charge carriers into said N−GaN drift layer and said drain is configured to collect charge carriers flowing through said device, with said flow being indicative of the presence and concentration of said target molecules.

63. The vertical GaN semiconductor sensor of claim 55, wherein said pGaN layer and said N−GaN drift layer form a vertical charge carrier pathway optimized for high sensitivity in detecting molecular interactions.

64. The vertical GaN semiconductor sensor of claim 55, wherein said depletion layers operate to fully form and prevent charge carriers from moving freely across said sensor in the absence of said target molecules, thereby setting a baseline conductance state of the sensor.

65. The vertical GaN semiconductor sensor of claim 55, wherein said binding events between said capture molecules and said target molecules induce changes in said electric field at the interface of said pGaN and said N−GaN drift layers, detected as variations in current flow between said source and said drain.

66-70. (canceled)
New Claims

71. A vertical semiconductor sensor comprising:
a wafer having a first charge carrier type forming a substrate;
a drift layer of opposite charge carrier type disposed over said wafer;
a junction forming layer disposed over said drift layer, forming a diode junctions therewith;
a drain located at a bottom portion of said wafer;
one or more sources disposed at a top surface of said junction forming layer;
one or more depletion layers formed at interfaces between said junction forming layer and said drift layer, configured to control charge carrier flow based on binding events occurring at detection areas;
capture molecules immobilized at a detection area formed adjacent to the one or more sources and configured to bind target molecules;
a gate electrode configured to apply a gate voltage to modulate a field effect at the depletion layers;
wherein said sensor is configured to detect target molecules by modulation of the depletion layers and charge carrier flow in response to binding events.

72. A vertical semiconductor sensor according to claim 71, further comprising, a plurality of capillary channels defined within said drift layer; wherein the capture molecules are immobilized within said capillary channels.

73. A vertical semiconductor sensor according to claim 71, wherein the gate electrode is a liquid gate electrode
configured to apply a gate voltage through a liquid sample disposed at said detection areas.

74. The vertical semiconductor sensor of claim 72 wherein said capillary channels are configured to guide a liquid sample to said detection areas for interaction with said capture molecules.

75. The vertical semiconductor sensor of claim 71, further comprising termination edges around said sensor elements to isolate electrical activity within each sensor and prevent electrical cross-talk between adjacent sensors.

76. The vertical GaN semiconductor sensor of claim 71, wherein said sources are configured to inject charge carriers into said N−GaN drift layer and said drain is configured to collect charge carriers flowing through said device, with said flow being indicative of the presence and concentration of said target molecules.

77. The vertical GaN semiconductor sensor of claim 71, wherein said junction forming layer and said drift layer form a vertical charge carrier pathway optimized for high sensitivity in detecting molecular interactions.
